(19) United States
(12) Patent Application Publication
(10) Pub. No.: US 2025/0269044 A1
(43) Pub. Date: Aug. 28, 2025

(54) PSMA-TARGETED DENDRIMER NANOPLATFORM FOR CANCER DETECTION AND THERAPY

(71) Applicant: Washington State University, Pullman, WA (US)

(72) Inventors: Anjali Sharma, Pullman, WA (US); Clifford Berkman, Pullman, WA (US)

(21) Appl. No.: 19/059,365

(22) Filed: Feb. 21, 2025

Related U.S. Application Data
(60) Provisional application No. 63/673,137, filed on Jul. 18, 2024.
(60) Provisional application No. 63/559,153, filed on Feb. 28, 2024.

(51) Int. Cl. A61K 47/59; A61K 47/54; A61K 47/61; A61K 47/68; A61P 35/00

(52) U.S. Cl. CPC A61K 47/595; A61K 47/54; A61K 47/61; A61K 47/68037; A61P 35/00

(57) ABSTRACT

Provided herein is a dendrimer complex comprising a dendrimer and a prostate specific membrane antigen (PSMA) ligand conjugated to an outer surface of the dendrimer. The dendrimer complex may further include one or more drugs, imaging agents, and/or radioligands conjugated to an outer surface of the dendrimer. Such complexes are useful in methods for detecting or inhibiting proliferation of PMSA-expressing cells and for methods of treating cancer characterized by PMSA expression.

### CROSS-REFERENCE TO RELATED APPLICATIONS

This application claims benefit of U.S. provisional patent applications 63/673,137 filed Jul. 18, 2024 and 63/559,153, filed Feb. 28, 2024, the contents of which are incorporated herein by reference.

### STATEMENT OF FEDERALLY SPONSORED RESEARCH AND DEVELOPMENT

This invention was made with government support under grant number R21 CA256382 awarded by the National Institutes of Health (NIH). The government has certain rights in the invention.

### FIELD OF THE INVENTION

The invention is generally related to dendrimers conjugated to a prostate specific membrane antigen (PSMA) ligand as a nanoplatform for cancer detection and therapy.

### BACKGROUND OF THE INVENTION

Prostate cancer (PCa) stands as one of the most prevalent forms of cancer and significantly contributes to male mortality worldwide. Although early-stage treatments show promising 5-year survival rates, the advanced disease has poor prognosis. Current standard of care for PCa involves androgen deprivation therapy, but most patients benefit from it for >24 months developing advanced disease. Treatment options for advanced PCa patients remain limited due to challenges associated with effective and selective drug delivery to PCa cells. Most chemotherapies are distributed throughout the body and often induce severe dose-dependent adverse effects owing to their off-target absorption within healthy organs and tissues. Hence, there exists a pressing requirement to develop platforms capable of selective intracellular delivery of potent chemotherapeutics to PCa cells, while exhibiting no or minimal side effects.

Significant progress has been made in the realm of the management of PCa, particularly focusing on prostate-specific membrane antigen (PSMA) targets for imaging and therapy. PSMA is a transmembrane glycoprotein that is highly expressed on prostatic epithelium and PCa cells, especially in advanced or metastatic stages, while showing reduced levels of expression on normal cells. Because of its high expression in tumor, it has become an appealing target for the creation of targeted chemotherapeutic agents and radio imaging tracers designed to identify and locate suspected metastases. Targeting PSMA is used clinically for imaging and radio-ligand therapy. Recently, the FDA approved the first PSMA-targeted radioligand therapy, ¹⁷⁷Lu-PSMA-617 (Pluvicto™). The major hurdle in the clinical success of small molecule PSMA probes and inhibitors is their poor pharmacokinetic profiles that limits their effectiveness. Pluvicto™ is guided by a reversible PSMA inhibitor (ACUPA), for treating PSMA-positive (PSMA+) advanced PCa. It has been reported that the PSMA ligands exhibiting an irreversible mode of binding demonstrate enhanced internalization in PSMA (+) cells in contrast to ligands with reversible mode of binding. However, inadequate pharmacokinetic profiles and poor drug loading capacities limit the efficacy and clinical utilities of PSMA inhibitors.

New nanoplatforms for selective intracellular delivery of potent chemotherapeutics to tumor cells are needed.

### SUMMARY

An aspect of the disclosure provides a dendrimer complex comprising a dendrimer having a neutral surface charge and a prostate specific membrane antigen (PSMA) ligand conjugated to an outer surface of the dendrimer. In some embodiments, the dendrimer is a generation 0-10 dendrimer. In some embodiments, the dendrimer is a polyamidoamine (PAMAM) dendrimer, such as a hydroxyl-terminating PAMAM dendrimer, or a 2-deoxyglucose (2DG) dendrimer. In some embodiments, the PSMA ligand is selected from the group consisting of CTT1298, ACUPA, and 2-PMPA.

The dendrimer complex may further comprise one or more drugs, imaging agents, and/or radioligands conjugated to an outer surface of the dendrimer. In some embodiments, the one or more drugs comprises an anti-cancer small molecule drug, such as a tyrosine kinase inhibitor, topoisomerase inhibitor, proteasome inhibitor, or matrix metalloproteinase inhibitor. In some embodiments, the tyrosine kinase inhibitor is cabozantinib or hydroxyl-cabozanitib. In some embodiments, the topoisomerase inhibitor is camptothecin.

Another aspect of the disclosure provides a pharmaceutical composition comprising a denrimer complex as described herein and a pharmaceutically acceptable carrier.

Another aspect of the disclosure provides a method of inhibiting proliferation of PMSA-expressing cells, comprising contacting the PMSA-expressing cells a dendrimer complex as described herein.

Another aspect of the disclosure provides a method of treating cancer characterized by expression of PMSA in a subject in need thereof, comprising administering to the subject a therapeutically effective amount of a dendrimer complex as described herein. In some embodiments, the cancer is prostate cancer.

Another aspect of the disclosure provides a method of detecting PMSA-expressing cells, comprising contacting the PMSA-expressing cells with a dendrimer complex as described herein, wherein the complex further comprises an imaging agent conjugated to an outer surface of the dendrimer; and detecting the imaging agent.

### BRIEF DESCRIPTION OF THE DRAWINGS

FIGS. 1A-B. Preparation and structural elucidation of PD-CTT1298-Cy5 conjugate. (A) Schematic representation for the synthesis of fluorescently labelled PSMA targeted dendrimer via SPAAC reactions. Reagents and conditions: (i) KHCO₃, ddH₂O, THF, RT, 3 H, 60%; (ii) 6-azido-hexanoic acid, EDC.HCl, DMAP, Anhy. DMF, RT, 24 H, 82%; (iii) DI H₂O, 12 H, RT, 92%; (iii) DI H₂O, 48 H, RT, 81%. (B) ¹H NMR spectra showing characteristic proton signals of CTT1298, Cy5 and PD at each step of the synthesis.

FIGS. 2A-C. Physicochemical characterization of PD-CTT1298-Cy5 conjugate and intermediates. (A) HPLC chromatograms showing purity and shift in the retention time at different steps of synthesis and showing peak at 650 nm after successful conjugation of Cy5. (B) Hydrodynamic radius of PD-CTT1298 analyzed by dynamic light scattering (DLS) in triplicates. (C) Physicochemical properties of PD-CTT1298-Cy5 dendrimer. Size and zeta potential are presented for non-fluorescent PD-CTT1298.

FIGS. 3A-D. Selective Uptake observed with PD-CTT-1298-Cy5 in PSMA (+) PC3-PIP cells. (A) Confocal microscopy images from PSMA (+) PC3-PIP and PSMA (−) PC3 cells incubated with 50 μg/mL (2.5 μM) of the fluorescently labeled PSMA targeted dendrimer, PD-CTT1298-Cy5. (B) Mean Fluorescence Intensities (MFI) of PC3-PIP and PC3 cells loaded with PD-CTT1298-Cy5 dendrimer over 2 H. (C) MFI of PC3-PIP cells incubated with and without a PSMA irreversible inhibitor, CTT 1057 (blocking agent), prior to loading with PD-CTT1298-Cy5 dendrimer over 2 H. (D) Dose response at 1H with PSMA (+) PC3-PIP cells. Data representative of experiments performed in triplicate. The p-values were calculated between the PC3-PIP cells and PC3 cells in the uptake experiments including with and without the addition of CTT 1057 at each time point in the blocking experiments with * p<0.05, ** p<0.01 and *** p<0.001.

FIGS. 4A-B. In vivo and ex vivo fluorescence imaging of PSMA-targeted dendrimer for tumor targeting and whole-body biodistribution. (A) Quantitative analysis of in vivo fluorescence signals from the tumors at 6 H, 24 H, and 48 H (n=3). (B) Quantitative analyses of ex vivo fluorescence signals from the organ tissues and tumors (n=3). The data was presented as mean±S.D.

FIGS. 5A-B. Quantitative tumor and organ biodistribution of PD-CTT1298-Cy5 at 48 H after systemic administration in PC3-PIP tumor xenograft mouse model. Quantitative biodistribution of PD-CTT1298-Cy5 in (A) tumour, and (B) all major organs at 24 h time point (n=3). The data was obtained through fluorescence spectroscopy of homogenized tissue extracts containing PD-CTT1298-Cy5 and reported as a percentage of the injected dose in total organ.

FIGS. 6A-B. Synthesis and characterization of PD-CTT1298-Cabo conjugate. (A) Schematic representation of the conjugation of Cabo and PSMA ligand on PAMAM-G4-OH to synthesize PD-CTT1298-Cabo conjugate; Reagents and conditions: (i) NaH, DMF, 80° C., 16h, 92%; (ii) HATU, DIPEA, DMF, RT, 16 h, 90%; (iii) TFA, 60° C., 30 min, 87%; (iv) DBCO acid, EDC.HCl, DMAP, DCM, 2 h, RT, 57%; (v) 6-azido-hexanoic acid, EDC.HCl, DMAP, anhy. DMF, RT, 24 h, 86%; (vi) DMF, 3 h, RT, 88%; (vii) DI water, 3 h, RT, 91% (B) ¹H NMR spectra of PD-CTT1298-Cabo dendrimer and intermediates representing the appearance and disappearance of characteristic protons.

FIGS. 7A-D. Characterization of PD-CTT1298-Cabo conjugates. (A) HPLC traces of intermediates and PD-CTT1298-Cabo conjugate demonstrating a shift in retention time at each synthetic step. (B) In vitro drug release profile of PD-CTT1298-Cabo conjugate at plasma and intra-tumoral conditions at 37° C. (C) Table showing the physicochemical properties of PD-CTT1298-Cabo conjugate. (D) In vitro efficacy of PD-CTT1298-Cabo in comparison with free Cabo and Cabo-OH over 72 H in PC3-PIP PSMA (+) cells. Cell viability (%) was calculated using the luminescence values obtained from the controls used in the experiment. Data representative of experiments performed in triplicate. p-values were calculated between Cabo, Cabo-OH, and the PD-CTT1298-Cabo dendrimer at each concentration tested with * p<0.05, ** p<0.01 and *** p<0.001.

FIG. 8. Chemical structure of 2DG-D dendrimer.

FIG. 9. Synthesis of PSMA-2DG-D-Cy5 and PSMA-2DG-D-Cabo conjugates. Schematic representation for the synthesis of dendrimer conjugates. Reagents and conditions: (i) 6-azido-hexanoic acid, EDC.HCl, DMAP, Anhy. DMF, RT, 24 h, 85% (2a) and 82% (2b); (ii) 12 h, RT, 90%; (iii) 48 h, RT, 81%; (iv) DBCO acid, EDC.HCl, DMAP, DCM, 2 h, RT, 57%; (v) DMF, 3 h, RT, 88%; (vi) 48 h, RT, 91%.

FIGS. 10A-B. The characterization of PSMA-2DG-D-Cy5. (A) Stacked ¹H NMR spectra showing successful stepwise synthesis of intermediates and PSMA-2DG-D-Cy5. (B) HPLC chromatogram showing considerable shift in retention time of 2DG-Azide, DBCO-C6-CTT1298, PSMA-2DG-D, and PSMA-2DG-D-Cy5.

FIGS. 11A-B. The characterization of PSMA-2DG-D-Cabo. (A) Stacked ¹H NMR spectra showing successful stepwise synthesis of intermediates and final dendrimer-drug conjugate. (B) HPLC chromatogram showing considerable shift in retention time of DBCO-Cabo, 2DG-D-Azide, 2DG-D-Cabo, DBCO-C6-CTT1298, and PSMA-2DG-D-Cabo.

FIGS. 12A-D. Physiochemical characterization of PSMA-2DG-D-Cy5 and PSMA-2DG-D-Cabo conjugates. (A) Physicochemical properties of PSMA-2DG-D-Cabo. (B) 2DG-D-Cabo demonstrates several folds higher water solubility than free Cabo. (C) Size distribution of PSMA-2DG-D and PSMA-2DG-D-Cabo. (D) Zeta potential distribution of PSMA-2DG-D and PSMA-2DG-D-Cabo as analyzed by dynamic light scattering (DLS) in triplicates.

FIGS. 13A-C. Selective uptake of PSMA-2DG-D-Cy5 in PSMA (+) PC3-PIP cells via PSMA mediated internalization. Quantitative uptake via flow cytometry. (A) Mean Fluorescence Intensities (MFI) of PC3-PIP and PC3 cells loaded with 1 μg/ml (50 nM) PSMA-2DG-D-Cy5 dendrimer over 1H. (B) Mean Fluorescence Intensities (MFIs) of PC3-PIP cells incubated with and without a PSMA irreversible inhibitor, CTT 1057 (blocking agent), prior to loading with PSMA dendrimer over 1H. The concentration of CTT 1057 was 10-fold higher than the concentration of the dendrimer tested in this experiment. (C) Dose response at 1H with various concentrations of the dendrimer in PC3-PIP PSMA (+) cells. Data representative of experiments performed in triplicate. Additionally, a blank subtraction was performed. p-values were calculated between the PC3-PIP cells and PC3 cells in the uptake experiments, with and without the addition of CTT 1057 at each time point in the blocking experiments, and between each concentration and cells without the dendrimer with * p<0.05, ** p<0.01 and *** p<0.001.

FIGS. 14A-C. In vitro drug release and efficacy of PSMA-2DG-D-Cabo conjugates. (A, B) Drug release study at physiological temperature under plasma and intracellular conditions. PSMA-2DG-D-Cabo demonstrates a sustained drug release profile at intra-tumoral conditions and is stable at plasma conditions. (C) Cell viability experiment with PSMA-2DG-D-Cabo dendrimer conjugate, free Cabo and Cabo-OH over 72 h in PSMA (+) PC3-PIP cells. Cell viability (%) was calculated using the luminescence values obtained and normalized with values from positive (+) control (10% DMSO) and negative (−) control (untreated cells) cohort used in the experiment. Data representative of experiments performed in triplicate. EC₅₀ values were calculated using Nonlinear regression with variable slope (PSMA-2DG-D-Cabo R²: 0.9604, Cabo-OH R²: 0.9849, Cabo R²: 0.9260). p-values were calculated between Cabo-OH and the PSMA-2DG-D-Cabo dendrimer as well as between free Cabo and the dendrimer conjugate at each concentration tested with *p<0.05, **p<0.01 and ***p<0.001.

FIGS. 15A-B. In vivo and ex vivo fluorescence imaging for tumor targeting and biodistribution. (A) Analyses of fluorescent signals from the tumors. Data represent the mean±SD. *p<0.05, **p<0.01. (B) Analyses of ex vivo fluorescent signals from the major organs and tumors. Data represent the mean±SD. *p<0.05, **p<0.01.

FIG. 16. Schematic representation for the synthesis of Tre-D-Dasa dendrimer via CuAAC and SPAAC reaction.

### DETAILED DESCRIPTION

Embodiments of the disclosure provide compositions and methods for cancer detection and therapy using dendrimers conjugated to a prostate specific membrane antigen (PSMA) ligand.

A dendrimer is a synthetic highly branched monodisperse and polyfunctional macromolecule, constituted by repetitive units (so-called “generations”) that are chemically bound to each other by an arborescent process around a multifunctional central core. Dendrimers can be considered to have three major portions: a core, an inner shell, and an outer shell. Exemplary chemical moieties for the core, inner shell, and outer shell are independently selected from dipentaerythritol, pentaerythritol, 2-(aminomethyl)-2-(hydroxymethyl) propane-1,3-diol, 2-ethyl-2-(hydroxymethyl) propane-1,3-diol, 3,3′,3″,3′″-silanetetrayltetrakis (propane-1-thiol), 3,3-divinylpenta-1,4-diene, 3,3′,3″-nitrilotripropionic acid, 3,3′,3″-nitrilotris (N-(2-aminoethyl)propanamide), 3,3′,3″,3′″-(ethane-1,2-diylbis(azanetriyl)) tetrapropanamide, 3-(carboxymethyl)-3-hydroxypentanedioic acid, 2,2′-((2,2-bis((2-hydroxyethoxy)methyl) propane-1,3-diyl)bis(oxy))bis(ethan-1-ol), tetrakis(3-(trichlorosilyl) propyl)silane, 1-Thioglycerol, 2,2,4,4,6,6-hexachloro-1,3,5,215,415,615-triazatriphosphinine, 3-(hydroxymethyl)-5,5-dimethylhexane-2,4-diol, 4,4′,4″-(ethane-1,1,1-triyl)triphenol, 2,4,6-trichloro-1,3,5-triazine, 5-(hydroxymethyl) benzene-1,2,3-triol, 5-(hydroxymethyl)benzene-1,3-diol, 1,3,5-tris(dimethyl(vinyl)silyl) benzene, Carbosiloxane core, nitrilotrimethanol, ethylene diamine, propane-1,3-diamine, butane-1,4-diamine, 2,2′,2″-nitrilotris (ethan-1-ol), alpha cyclodextrin, beta cyclodextrin, gamma cyclodextrin, benzene-1,2,3,4,5,6-hexathiol, monosaccharide, disaccharides, trisaccharides, oligosaccharides, chitosan, and derivatives thereof.

The term “dendrimer” includes, but is not limited to, a molecular architecture with an interior core and layers (or “generations”) of repeating units which are attached to and extend from this interior core, each layer having one or more branching points, and an exterior surface of terminal groups attached to the outermost generation. In some embodiments, dendrimers have regular dendrimeric or “starburst” molecular structures. Generally, dendrimers have a diameter from about 1 nm up to about 50 nm, such as from about 1-5, 5-10, 10-15, 15-20, 20-25, 25-30, 35-40, 40-45 or 45-50 nm in diameter, including all single digits within these ranges.

Applications of dendrimers typically involve conjugating other chemical species to the dendrimer surface that can function as detecting agents (such as a dye molecule), affinity ligands, targeting components, radioligands, imaging agents, or pharmaceutically active compounds. As described herein, dendrimers conjugated to PSMA ligands may be used as a nanoplatform to target anticancer agents to PSMA-expressing cells/tumors thus enhancing drug effectiveness while mitigating dose-related toxicity and systemic side effects.

Drug attachment to the dendrimer may be accomplished by (1) a covalent attachment or conjugation to the external surface of the dendrimer forming a dendrimer prodrug, (2) ionic coordination to charged outer functional groups, or (3) micelle-like encapsulation of a drug via a dendrimer-drug supramolecular assembly.

Dendrimers are also classified by generation, which refers to the number of repeated branching cycles that are performed during its synthesis. For example, if a dendrimer is made by convergent synthesis, and the branching reactions are performed onto the core molecule three times, the resulting dendrimer is considered a third generation dendrimer. Each successive generation results in a dendrimer roughly twice the molecular weight of the previous generation. Dendrimers may have a single surface functional group, or may be modified to allow for multiple functional groups on the surface.

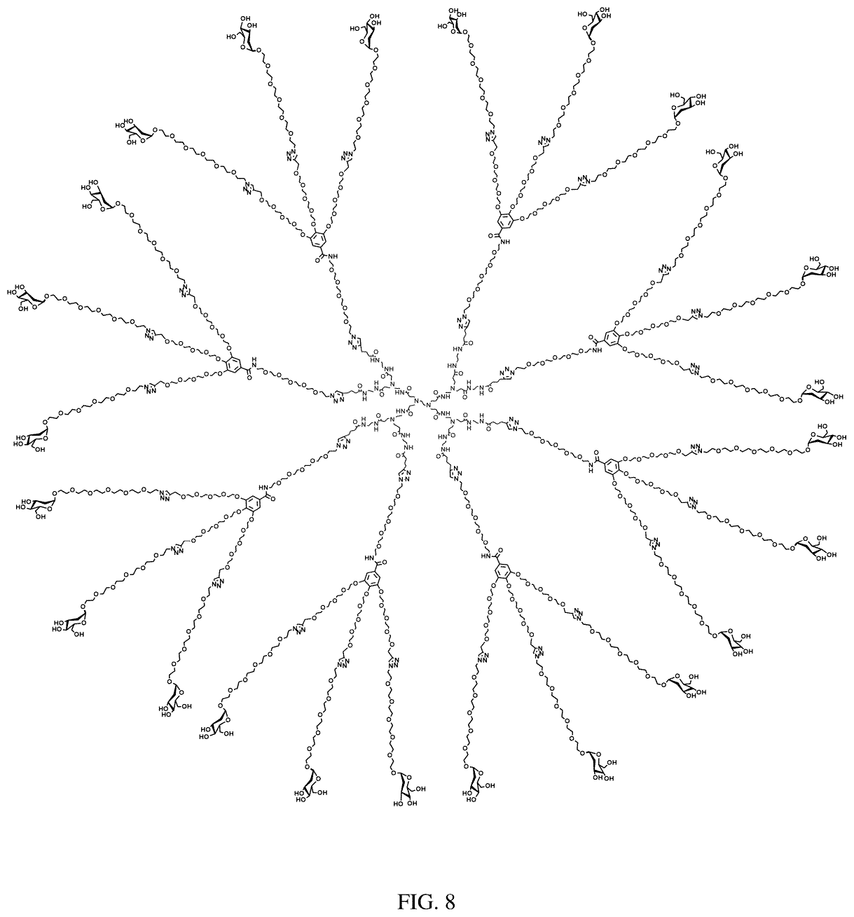

Suitable dendrimers may be generation 0 to generation 10. In preferred embodiments, the dendrimer is a generation 2, 3, 4, 5 or 6 dendrimer. In some embodiments, the dendrimers are not cationic (i.e. the dendrimers described herein have a neutral or anionic surface charge). In some embodiments, the dendrimers may be amine-terminating or hydroxyl-terminating dendrimers. Dendrimers may be composed of mixed layers or identical layers. Suitable dendrimers include phosphorous dendrimers, peptide dendrimers, polyamidoamine (PAMAM) dendrimers, polypropyleneimine (PPI) dendrimers, polyethyleneimine (PEI) dendrimers, polyethylene glycol-based dendrimers, polyester dendrimers, polylysine dendrimers, polypropylamine (POPAM) dendrimers, iptycene dendrimers, aliphatic poly (ether) dendrimers, aromatic polyether dendrimers, micellar dendrimers, and glycodendrimers such as 2-deoxyglucose (2DG) dendrimer. A glycodendrimer may encompass (1) carbohydrate-coated; (2) carbohydrate-centered; or (3) carbohydrate-based dendrimers. For 2DG dendrimer, the innermost layer (the core) comprises alkyne-terminating generation-1 PAMAM dendrimer, followed by a second layer composed of gallic acid building blocks, and the outermost layer comprises 2-DG (FIG. 8).

The molecular weight of the dendrimers can be varied to prepare polymeric nanoparticles that form particles having properties, such as drug release rate, optimized for specific applications. The dendrimers can have a molecular weight of between about 150 Da and 1 MDa. In certain embodiments, the polymer has a molecular weight of between about 500 Da and about 100 kDa, more preferably between about 1 kDa and about 50 kDa, most preferably between about 1 kDa and about 20 kDa.

Dendrimers may have a certain surface density of particular functional groups (e.g. hydroxyl or amine groups). For example, the dendrimer may have a surface density of a certain functional group of at least 1 functional group/nm² (number of surface groups/surface area in nm²). For example, in some embodiments, the surface density of certain functional groups (e.g. hydroxyl or amine groups) is between about 1 and about 50, e.g. more than 2, 3, 4, 5, 6, 7, 8, 9, 10, 15, 20, 25, 30, 35, 40, 45, 50, or more than 50 group/nm².

In some embodiments, the dendrimers may have a fraction of the preferred functional groups exposed on the outer surface, with the others in the interior core of the dendrimers. For example, the dendrimers have a volumetric density of certain functional groups of at least 1 functional group/nm³ (number of functional groups/volume in nm³). For example, in some embodiments, the volumetric density of hydroxyl groups is between about 1 and about 50, e.g. more than 2, 3, 4, 5, 6, 7, 8, 9, 10, 15, 20, 25, 30, 35, 40, 45, 50, or more than 50 group/nm³.

Dendrimers may be prepared by methods known in the art. Dendritic structures are mostly synthesized by two different approaches: divergent or convergent. Many other synthetic pathways exist, such as the orthogonal approach, accelerated approaches, the double-stage convergent method or the hypercore approach, the hypermonomer method or the branched monomer approach, the double exponential method; the orthogonal coupling method or the two-step approach, the two monomers approach, or the AB₂-CD₂ approach.

The core of the dendrimer, one or more branching units, one or more linkers/spacers, and/or one or more surface groups can be modified to allow conjugation to further functional groups (branching units, linkers/spacers, surface groups, etc.), monomers, targeting agents, and/or active agents via click chemistry. “Click chemistry” involves, for example, the coupling of two different moieties (e.g., a core group and a branching unit; or a branching unit and a surface group) via a 1,3-dipolar cycloaddition reaction between an alkyne moiety (or equivalent thereof) on the surface of the first moiety and an azide moiety (e.g., present on a triazine composition) (or equivalent thereof) (or any active end group such as, for example, a primary amine end group, a hydroxyl end group, a carboxylic acid end group, a thiol end group, etc.) on the second moiety.

For targeting to PSMA-expressing cells/tumors, an irreversible or reversible PSMA ligand is conjugated to an outer surface of the dendrimer. As used herein, “irreversible” means that the PSMA ligand binds to PSMA with a Kd in the nanomolar range under physiological conditions, such as at pH values of from about 7.0-7.5, e.g. from about 7.35 to 7.45. An irreversible PSMA ligand binds to PSMA and does not dissociate under physiological conditions. In contrast, a “reversible” PSMA ligand may bind non-covalently and can readily detach from the target allowing for the ligand to be released and potentially re-bind to another PSMA molecule.

The PSMA ligand is “conjugated” to a functional group on the outer surface of the dendrimer, generally a group that is surface exposed and available to react with the PSMA ligand. “Conjugated” as used herein refers to covalently bonded to a surface functional group of the ligand.

The dendrimers employed herein generally exhibit a surface charge that is neutral (uncharged). That is to say, the functional groups on the surface of the dendrimer, e.g. hydroxyl, acetyl, or other polar uncharged groups, provide a neutral (not cationic or anionic) surface charge. In general, the dendrimer has a zeta potential value that is close to zero, e.g. within a range of −10 mV to +10 mV.

In some embodiments, the ligand is a phosphoramidate-based PSMA inhibitor. In some embodiments, the PSMA ligand is selected from the group consisting of CTT1298, ACUPA, 2-PMPA, CTT1057, ^{99m} Tc-MIP-1404, MIP-1072, MIP-1095, 177Lu-PSMA-617, PSMA-617, 225Ac-PSMA-617, PSMA-1007, PSMA-11, PSMA-I&T, ¹⁸F-PSMA-1007, DCFPyL, 18F-rhPSMA-7.3, and 68Ga-PSMA-11. Suitable PSMA ligands may also include urea-based glutamate heterodimers, phosphoramidates, and 2-(phosphinylmethyl) pentanedioic acids. Generally, the PSMA ligand is conjugated to an outer surface of the dendrimer at a concentration of about 0.01% to about 20%, preferably about 1% to about 20%, more preferably about 5% to about 20% by weight.

In some embodiments, in addition to the PSMA ligand, one or more agents, such as one or more drugs, imaging agents, and/or radioligands, are covalently attached to the dendrimers. In some embodiments, the agents are attached to the dendrimer via a linking moiety that is designed to be cleaved in vivo. The linking moiety can be designed to be cleaved hydrolytically, enzymatically, or combinations thereof, so as to provide for the sustained release of the active agents in vivo. Both the composition of the linking moiety and its point of attachment to the active agent, are selected so that cleavage of the linking moiety releases either an active agent, or a suitable prodrug thereof. The composition of the linking moiety can also be selected in view of the desired release rate of the active agents.

In some embodiments, the attachment occurs via one or more of disulfide, ester, ether, thioester, carbamate, carbonate, hydrazine, or amide linkages. In preferred embodiments, the attachment occurs via an appropriate spacer that provides a disulfide bridge between the agent and the dendrimer. In this case, the dendrimer complexes are capable of rapid release of the agent in vivo by thiol exchange reactions, under the reduced conditions found in body.

Linking moieties generally include one or more organic functional groups. Examples of suitable organic functional groups include secondary amides (—CONH—), tertiary amides (—CONR—), secondary carbamates (—OCONH—; —NHCOO—), tertiary carbamates (—OCONR—; —NRCOO—), ureas (—NHCONH—; —NRCONH—; —NHCONR—, —NRCONR—), carbinols (—CHOH—, —CROH—), disulfide groups, hydrazones, hydrazides, ethers (—O—), and esters (—COO—, —CH₂O₂C—, CHRO₂C—), wherein R is an alkyl group, an aryl group, or a heterocyclic group. In general, the identity of the one or more organic functional groups within the linking moiety can be chosen in view of the desired release rate of the active agents.

In certain embodiments, the linking moiety includes one or more of the organic functional groups described above in combination with a spacer group. The spacer group can be composed of any assembly of atoms, including oligomeric and polymeric chains; however, the total number of atoms in the spacer group is preferably between 3 and 200 atoms, more preferably between 3 and 150 atoms, more preferably between 3 and 100 atoms, most preferably between 3 and 50 atoms. Examples of suitable spacer groups include alkyl groups, heteroalkyl groups, alkylaryl groups, oligo- and polyethylene glycol chains, and oligo-and poly (amino acid) chains.

A wide range of agents may be associated with the dendrimers to be delivered. The agents can be proteins or peptides, sugars or carbohydrate, nucleic acids or oligonucleotides, lipids, small molecules, or combinations thereof. The nucleic acid can be an oligonucleotide encoding a protein, for example, a DNA expression cassette or an mRNA. Representative oligonucleotides include siRNAs, microRNAs, DNA, and RNA. In some embodiments, the active agent is a therapeutic antibody. One or more types of active agents can be encapsulated, complexed or conjugated to the dendrimer.

Exemplary therapeutic agents include chemotherapeutics, anti-inflammatory drugs, antiproliferatives, vasodilators, neuroactive agents and anti-infective agents. In some embodiments, the dendrimer is linked to the targeting moiety, imaging agents, and/or therapeutic agents via a spacer ending in disulfide, ester, ether, thioester, carbamate, carbonate, hydrazine, or amide bonds. Generally, one or more prophylactic, therapeutic, and/or diagnostic agents are encapsulated, associated, and/or conjugated in the dendrimer complex at a concentration of about 0.01% to about 30%, preferably about 1% to about 20%, more preferably about 5% to about 20% by weight. The dendrimer can be conjugated to more than one agent and more than one type of agent.

Chemotherapeutic agents generally include pharmaceutically or therapeutically active compounds that work by interfering with DNA synthesis or function in cancer cells. Based on their chemical action at a cellular level, chemotherapeutic agents can be classified as cell-cycle specific agents (effective during certain phases of cell cycle) and cell-cycle nonspecific agents (effective during all phases of cell cycle). Examples of chemotherapeutic agents include tyrosine kinase inhibitors, topoisomerase inhibitors, proteasome inhibitors, matrix metalloproteinase inhibitors, alkylating agents, angiogenesis inhibitors, aromatase inhibitors, antimetabolites, anthracyclines, antitumor antibiotics, platinum drugs, radioactive isotopes, radiosensitizing agents, checkpoint inhibitors, PD1 inhibitors, plant alkaloids, glycolytic inhibitors, and prodrugs thereof.

Representative chemotherapeutic agents include, but are not limited to, cabozantinib, hydroxyl-cabozanitib, camptothecin, amsacrine, bleomycin, busulfan, capecitabine, carboplatin, carmustine, chlorambucil, cisplatin, cladribine, clofarabine, crisantaspase, cyclophosphamide, cytarabine, dacarbazine, dactinomycin, daunorubicin, docetaxel, doxorubicin, epipodophyllotoxins, epirubicin, etoposide, etoposide phosphate, fludarabine, fluorouracil, gemcitabine, hydroxycarb amide, idarubicin, ifosfamide, innotecan, leucovorin, liposomal doxorubicin, liposomal daunorubici, lomustine, mechlorethamine, melphalan, mercaptopurine, mesna, methotrexate, mitomycin, mitoxantrone, oxaliplatin, paclitaxel, pemetrexed, pentostatin, procarbazine, raltitrexed, satraplatin, streptozocin, teniposide, tegafur-uracil, temozolomide, teniposide, thiotepa, tioguanine, topotecan, treosulfan, vinblastine, vincristine, vindesine, vinorelbine, taxol and derivatives thereof, trastuzumab (HERCEPTIN®), cetuximab, and rituximab (RITUXAN® or MABTHERA®), bevacizumab (AVASTIN®), and combinations thereof.

Dendrimer nanoparticles can include diagnostic agents useful for determining the location of administered particles. These agents can also be used prophylactically. Exemplary diagnostic materials include paramagnetic molecules, fluorescent compounds, magnetic molecules, and radionuclides. Suitable diagnostic agents include, but are not limited to, x-ray imaging agents and contrast media. Radionuclides also can be used as imaging agents.

Exemplary radioactive label include ¹⁴C, ³⁶C1, ⁵⁷Co, ⁵⁸Co, ⁵¹Cr, ¹²51, ¹³¹1, ¹¹¹Ln, ¹⁵²Eu, ⁵⁹Fe, ⁶⁷Ga, ³²P, ¹⁸⁶Re, ³⁵S, ⁷⁵Se, ¹⁷⁵Yb. Examples of other suitable contrast agents include gases or gas emitting compounds, which are radioopaque. In some embodiments, the imaging agent to be incorporated into the dendrimer nanoparticles is a fluorophore (e.g., fluorescein isothiocyanate (FITC), phycoerythrin (PE)), an enzyme (e.g., alkaline phosphatase, horseradish peroxidase), element particles (e.g., gold particles).

Embodiments provide methods for treating cancer by the administration of a composition as described herein. As used herein “treating” or “treatment” means any manner of managing the cancer by medicinal or other therapies, such that the cancer no longer increases in size, metastasizes, or otherwise progresses in severity on a diagnosis scale, such as Duke's classification or any other classification system known. In some embodiments, the treatment ameliorates the disease through a reduction in size or otherwise beneficially improves the severity on a diagnosis scale.

The compositions and methods are useful for treating subjects having benign or malignant tumors by delaying or inhibiting the growth of a tumor in a subject, reducing the growth or size of the tumor, inhibiting or reducing metastasis of the tumor, and/or inhibiting or reducing symptoms associated with tumor development or growth.

As used herein, the term “cancer” refers to a neoplasm, cancer, or precancerous lesion. The neoplasm or cancer may be benign or malignant. This includes cells or tissues that have characteristics relating to changes that may lead to malignancy or cancer, such as mutations controlling cell growth and proliferation. As the dendrimers include PSMA ligand for targeting, the cancer to be treated is characterized by expression of PSMA. In some embodiments, the cells/tumor have enhanced PSMA expression as compared to a healthy subject without cancer, e.g. at least 2-2000-fold higher expression. Examples of PSMA-expressing cancers to be treated include but are not limited to: prostate cancer, renal cell carcinoma, hepatocarcinoma, salivary gland cancer, glioblastoma, colon cancer, lung cancer, breast cancer, and bladder cancer.

The dendrimer complexes can be administered in combination with one or more additional therapeutically active agents, which are known to be capable of treating conditions or diseases discussed above.

In some embodiments the disclosure provides a method of treating cancer, wherein the method comprises determining if the subject has a PSMA-expressing cancer and if the subject is determined to have the PSMA-expressing cancer, then administering to the subject a therapeutically effective dose of a composition as disclosed herein.

The compositions described herein may be administered in vivo by any suitable route (e.g. parenterally or enterally) including but not limited to: inoculation or injection (e.g. intravenous, intraperitoneal, intramuscular, subcutaneous, intra-aural, intraarticular, intramammary, and the like), topical application, and by absorption through epithelial or mucocutaneous linings (e.g., nasal, oral, vaginal, rectal, gastrointestinal mucosa, and the like). Other suitable means include but are not limited to: inhalation (e.g. as a mist or spray), orally (e.g. as a pill, capsule, liquid, etc.), intravaginally, intranasally, rectally, by ingestion of a food or probiotic product containing the compound, as eye drops, etc. In preferred embodiments, the mode of administration is oral or by injection. In some embodiments, the treatment described herein is administered with or without radiation therapy.

A patient or subject to be treated by any of the compositions or methods of the present disclosure can mean either a human or a non-human animal including, but not limited to mammals, dogs, horses, cats, rabbits, gerbils, hamsters, rodents, birds, aquatic mammals, cattle, pigs, camelids, and other zoological animals.

In some embodiments, the formulation or active agent is administered to the subject in a therapeutically effective amount. By a “therapeutically effective amount” or an “effective amount” is meant a sufficient amount to treat the disease or disorder at a reasonable benefit/risk ratio applicable to any medical treatment. It will be understood that the total daily usage of the compositions of the present disclosure will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific active agent employed; and like factors well known in the medical arts. In the case of cancer, the effective amount of the drug or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer. It is well within the skill of the art to start doses of the compound at levels or frequencies lower than those required to achieve the desired therapeutic effect and to gradually increase the dosage or frequency until the desired effect is achieved. However, the daily dosage of the active agent may be varied over a wide range from 1 to 1,500 mg per adult per day. In particular, the compositions contain at least or up to 1.0, 2.5, 5.0, 10.0, 15.0, 25.0, 50.0, 100, 250, 500, 750, 1000, 1250, or 1500 mg of the active ingredient for the symptomatic adjustment of the dosage to the subject to be treated. A medicament typically contains from about 0.01 mg to about 1500 mg of the active ingredient, in particular from 1 mg to about 250 mg of the active ingredient. An effective amount of the drug is ordinarily supplied at a dosage level at least or up to 1 mg/kg to 100 mg/kg of body weight per day, e.g. about 5 mg/kg, 10 mg/kg, 15 mg/kg, 20 mg/kg, 30 mg/kg, 40 mg/kg, 50 mg/kg, 60 mg/kg, 70 mg/kg, 80 mg/kg, 90 mg/kg, or 100 mg/kg of body weight per day. Such doses may be administered in a single dose or it may be divided into multiple doses.

Further embodiments provide a method of a method of inhibiting proliferation of PMSA-expressing cells in vitro or in vivo, comprising contacting the PMSA-expressing cells a composition as described herein.

Further embodiments provide a method of detecting PMSA-expressing cells in vitro or in vivo, comprising contacting the PMSA-expressing cells with a complex comprising a dendrimer as described herein, a prostate specific membrane antigen (PSMA) ligand, and an imaging agent, wherein the PSMA ligand and imaging agent are conjugated to an outer surface of the dendrimer; and detecting the imaging agent.

Embodiments of the disclosure also provide pharmaceutical compositions comprising the dendrimer complexes described herein and a pharmaceutically acceptable carrier/excipient. The pharmaceutical compositions can be formulated according to known methods for preparing pharmaceutically useful compositions. The active ingredients may be mixed with excipients which are pharmaceutically acceptable and compatible with the active ingredients. Pharmaceutically” or “pharmaceutically acceptable” refer to molecular entities and compositions that do not produce an adverse, allergic or other untoward reaction when administered to a mammal, e.g. a human, as appropriate. As used herein, the phrase “pharmaceutically acceptable carrier” means any of the standard pharmaceutically acceptable carriers. The pharmaceutically acceptable carrier can include diluents, adjuvants, and vehicles, as well as implant carriers, and inert, non-toxic solid or liquid fillers, diluents, or encapsulating material that does not react with the active ingredients of the invention. Examples include, but are not limited to, phosphate buffered saline, physiological saline, water, and emulsions, such as oil/water emulsions. The carrier can be a solvent or dispersing medium containing, for example, ethanol, polyol (for example, glycerol, propylene glycol, liquid polyethylene glycol, and the like), suitable mixtures thereof, and vegetable oils. Formulations are described in a number of sources that are well known and readily available to those skilled in the art. For example, Remington's Pharmaceutical Sciences (Martin E W Easton Pa., Mack Publishing Company, 19^{th} ed.) describes formulations which can be used in connection with the subject invention. The final amount of the compounds in the formulations may vary. However, in general, the amount in the formulations will be from about 0.01-99%, weight/volume.

The dendrimer complexes described herein include the pharmaceutically acceptable salts thereof. “Salts” or “pharmaceutically acceptable salts” refers to the relatively non-toxic, inorganic and organic acid addition salts, and base addition salts, of compounds of the present disclosure. These salts can be prepared in situ during the final isolation and purification of the compounds. In particular, acid addition salts can be prepared by separately reacting the purified compound in its free base form with a suitable organic or inorganic acid and isolating the salt thus formed.

Compositions as described herein may be prepared either as liquid solutions or suspensions, or as solid forms such as tablets, pills, granules, capsules, powders, ampoules, and the like. The liquid may be an aqueous liquid. Solid forms suitable for solution in, or suspension in, liquids prior to administration may also be prepared. Example dosage forms include a tablet, dragee, liquid, drop, capsule, caplet, gelcap, etc.

Formulations suitable for parenteral administration include, for example, aqueous sterile injection solutions, which may contain antioxidants, buffers, bacteriostats, and solutes which render the formulation isotonic with the blood of the intended recipient; and aqueous and nonaqueous sterile suspensions which may include suspending agents and thickening agents. The formulations may be presented in unit-dose or multi-dose containers, for example sealed ampoules and vials, and may be stored in a freeze dried (lyophilized) condition requiring only the condition of the sterile liquid carrier, for example, water for injections, prior to use. Extemporaneous injection solutions and suspensions may be prepared from sterile powder, granules, tablets, etc. It should be understood that in addition to the ingredients particularly mentioned above, the formulations of the subject invention can include other agents conventional in the art having regard to the type of formulation in question. The pharmaceutical composition can be adapted for various forms of administration. Administration can be continuous or at distinct intervals as can be determined by a person skilled in the art.

The compositions of the present disclosure may also contain other components such as, but not limited to, additives, adjuvants, buffers, tonicity agents, bioadhesive polymers, and preservatives. In any of the compositions of this disclosure, the mixtures are preferably formulated at about pH 5 to about pH 8. This pH range may be achieved by the addition of buffers to the composition. It should be appreciated that the compositions of the present disclosure may be buffered by any common buffer system such as phosphate, borate, acetate, citrate, carbonate and borate-polyol complexes, with the pH and osmolality adjusted in accordance with well-known techniques to proper physiological values.

An additive such as a sugar, a glycerol, and other sugar alcohols, can be included in the compositions of the present disclosure. Pharmaceutical additives can be added to increase the efficacy or potency of other ingredients in the composition. For example, a pharmaceutical additive can be added to a composition of the present disclosure to improve the stability of the bioactive agent, to adjust the osmolality of the composition, to adjust the viscosity of the composition, or for another reason, such as effecting drug delivery. Non-limiting examples of pharmaceutical additives of the present disclosure include sugars, such as, trehalose, mannose, D-galactose, and lactose.

In an embodiment, if a preservative is desired, the compositions may optionally be preserved with any well-known system such as benzyl alcohol with/without EDTA, benzalkonium chloride, chlorhexidine, Cosmocil® CQ, or Dowicil 200.

Before exemplary embodiments of the present invention are described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

The invention is further described by the following non-limiting examples which further illustrate the invention, and are not intended, nor should they be interpreted to, limit the scope of the invention.

### EXAMPLE 1

Prostate cancer (PCa) is the second leading cause of cancer-related deaths among men in the United States. Although early-stage treatments exhibit promising 5-year survival rates, the treatment options for advanced stage disease are constrained, with short survival benefits due to the challenges associated with effective and selective drug delivery to PCa cells. Even though targeting Prostate Specific Membrane Antigen (PSMA) has been extensively explored and is clinically employed for imaging and radio-ligand therapy, the clinical success of PSMA-based approaches for targeted delivery of chemotherapies remains elusive. In this study, we combine a generation 4 hydroxy polyamidoamine dendrimer (PD) with irreversible PSMA ligand (CTT1298) to develop a PSMA-targeted nanoplatform (PD-CTT1298) for selective intracellular delivery of potent chemotherapeutics to PCa. PD-CTT1298-Cy5 exhibits a PSMA IC₅₀ in nanomolar range and demonstrates selective uptake in PSMA (+) PCa cells via PSMA mediated internalization. When systemically administered in a prostate tumor xenograft mouse model, PD-CTT1298-Cy5 selectively targets PSMA (+) tumors with significantly less accumulation in PSMA (−) tumors or upon blocking of the PSMA receptors. Moreover, the dendrimer clears rapidly from the off-target organs limiting systemic side-effects. Further, the conjugation of anti-cancer agent, cabozantinib to PSMA-targeted dendrimer translates to a significantly enhanced anti-proliferative activity in vitro compared to the free drug. These findings highlight the use of PD-CTT1298 nanoplatform as a versatile approach for selective delivery of high payloads of potent chemotherapeutics to PCa, where dose related systemic side-effects are a major concern.

### Experimental Section

### Synthesis of PD-CTT1298-Cy5 and PD-CTT1298-Cabo Conjugates

### Materials and Reagents

All starting materials and reagents were purchased from Sigma-Aldrich, Merck, or Thermo Fisher Scientific. The starting materials and solvents were used as received. Thin-layer chromatography was performed on a film of silica gel that contained a fluorescent indicator F₂₅₄ supported on an aluminum sheet (Merck). Column chromatography was performed using silica gel 60 (70-230 mesh) as the stationary phase. Dialysis was performed using Spectra/Por dialysis membranes purchased from Repligen.

### Experimental Instruments

¹H NMR (500 MHz), ¹³C NMR (125 MHz), and ³¹P NMR (202 MHz) spectra data were recorded on a Bruker 500 MHz spectrometer at 25° C. The samples were prepared in deuterated chloroform (CDCl₃), deuterated DMSO (DMSO-d₆) or deuterated water (D₂O). Chemical shifts (δ) are reported in parts per million (ppm) downfield by reference to proton resonances resulting from incomplete deuteration of the NMR solvent. Coupling constant (J) is reported in Hertz (Hz). Patterns of Splitting are designated as s: singlet, d: doublet, t: triplet, dd: double doublet, m: multiplet, ddd: doublet of doublet of doublet, and br: broad peak. High-Resolution Mass Spectra (HRMS) were recorded on a Bruker-micrOTOF-Q II spectrometer using ESI as the ion source. The purity and drug release studies were analyzed using high-performance liquid chromatography (HPLC). The HPLC was performed using a Waters Acquity Arc system (Milford, MA, USA), equipped with binary pumps, 2998 PDA detector, and a 2475 fluorescence detector. The analyses were performed using Waters Empower software. The samples were run using Waters C18 symmetry 300 (5 μm) 4.6×250 mm column using a gradient flow method. The method started with 90:10 (Solvent A: 0.1% TFA and 5% ACN in water; Solvent B: 0.1% TFA in ACN), gradually increased to 50:50 (A:B) at 20 minutes, 10:90 (A:B) at 38 minutes, and finally returned to 90:10 (A:B) at 40 minutes. A flow rate of 1 mL/min was maintained during the run. The dendrimers and drug were detected at 205, 210 or 305 nm. The Cy5 labeled conjugate was detected at 650 nm. The size and zeta potential distribution of dendrimers were determined using a Malvern Zetasizer Pro Blue (Malvern Panalytical) instrument. The samples for size distribution were dissolved in milli Q H₂O at a concentration of 0.1 mg/mL and the samples for zeta potential distribution were dissolved in 10 mM sodium chloride solution at a concentration of 0.2 mg/mL. Samples were measured in triplicate and averaged to provide the reported sizes and zeta potential.

### Synthetic procedures

Synthesis of compound 3: To a stirring of solution of CTT1298 (1) (0.27 g, 1.0 eq, 0.35 mmol) and KHCO₃ (0.047 g, 1.3 eq, 0.46 mmol) in ddH₂O (1.5 mL) was added a solution of DBCO-C6-NHS (0.1 g, 0.232 mmol) in THF (1.5 mL) dropwise. The reaction was stirred at ambient temperature for 3 hours, solvent was removed under reduced pressure and the resulting residue was purified via reverse-phase flash chromatography (100% ddH₂O→10% MeOH in ddH₂O) to yield a white solid in 60% yield.

¹H NMR (600 MHZ, D₂O) δ 7.69 (d, 1H), 7.55-7.40 (m, 6H), 7,34 (d, 1H), 5.11 (d, 1H), 4.17 (dd, 1H), 4.08 (dd, 1H), 3.82 (d, 1H), 3.74 (m, 2H), 3.49 (m, 1H), 3.11 (m, 2H), 2.38-2.06 (m, 10H), 2.01-1.79 (m, 6H), 1.71-1.58 (m, 5H), 1.5 (m, 2H), 1.36-1.14 (m, 6H); ¹³C NMR (101 MHz, D20) δ 183.0, 181.5, 179.2, 178.4, 176.3, 176.1, 175.7, 174.9, 150.8, 147.6, 131.9, 129.1, 129.0, 128.8, 128.4, 128.1, 127.0, 125.6, 122.3, 121.5, 114.6, 107.9, 64.4, 64.4, 56.6, 55.4, 55.4, 54.8, 39.1, 35.6, 35.2, 34.0, 33.7, 32.3, 32.0, 31.9, 28.1, 28.0, 27.8, 26.8, 26.7, 25.7, 24.9, 24.4, 24.2; ³¹P NMR (162 MHz, D₂O) δ 7.39. HRMS (MALDI) calcd [M−H]⁺ for C₄₂H₅₄N₅O₁₅P: 898.3276, found 898.3288

Synthesis of compound 5: PAMAM-G4-OH (PD) (1.0 g, 1.0 eq, 0.07 mmol) was dissolved in Anhy. DMF (5 mL). Azido Hexanoic Acid (77 mg, 7.0 eq, 0.49 mmol) was dissolved in anhy. DMF (5 mL) and was activated by adding EDC.HCl (134.5 mg, 10.0 eq, 0.7 mmol) and stirred for 15 minutes. It was then added dropwise to the solution of PD under continuous stirring followed by the addition of DMAP (38.5 mg, 5 eq, 0.32 mmol).

The reaction mixture was stirred for 24 hours at room temperature. The reaction completion was confirmed by the shift in the retention time of the chromatogram in HPLC. The dialysis was performed using a 1 kDa dialysis membrane in deionized water for 24 h. The aqueous solution was lyophilized to afford PD-azide (5) in 82% yield.

¹H NMR (500 MHz, DMSO) δ 8.15-7.73 (m, D-internal amide H), 4.76 (bs, D-OH), 4.04 (t, D-ester-CH₂), 3.50-3.25 (m, D-CH₂), 3.22-3.03 (m, D-CH₂), 2.77-2.59 (m, D-CH₂), 2.49-2.40 (m, D-CH₂), 2.35-2.13 (m, D-CH₂), 1.62-1.50 (linker-CH₂), 1.39-1.32 (linker-CH₂).

Synthesis of compound 6: To solution of PD-azide dendrimer (5) (20.0 mg, 1.0 eq, 0.0012 mmol) in DI Water (150 μL) was added DBCO C6 CTT1298 (3) (5.5 mg, 4.0 eq, 0.005 mmol), and the reaction mixture was stirred for 24 hours at room temperature. Reaction progress was tracked with HPLC. Upon completion, the dialysis was performed using a 1 kDa dialysis membrane in DI Water for 12 h. The aqueous solution was lyophilized to afford PD-CTT1298 (6) in 92% yield.

¹H NMR (500 MHz, D₂O) δ 7.72-7.13 (m, DBCO H), 4.18-3.92 (m, D-CH₂ and ligand H), 3.73-3.50 (m, D-CH₂ and ligand H), 3.45-3.14 (m, D-CH₂ and ligand H), 3.08-2.97 (m, linker-CH₂), 2.89-2.67 (m, D-CH₂), 2.66-2.49 (D-CH₂), 2.48-2.24 (m, D-CH₂), 2.25-2.10 (m, ligand H), 2.06-1.93 (m, ligand H), 1.90-1.70 (m, ligand H), 1.62-1.35 (m, linker-CH₂ and ligand H), 1.34-1.10 (m, linker —CH₂ and ligand H). ³¹P NMR (202 MHz, D₂O) δ 7.31.

Synthesis of compound 7: PD-(TT1298 (6) (19.0 mg, 1.0 eq, 0.0001 mmol) was dissolved in DI Water (150 μL) and stirred. Cy5-DBCO) (2.77 mg, 3.0 eq, 0.003 mmol) was added to the dendrimer solution and the stirring mixture was left for 48 hours at room temperature. The completion of reaction was tracked using HPLC. Upon completion, the dialysis was performed using a 1 kDa dialysis membrane in DI Water for 12 h. The aqueous solution was lyophilized to afford compound PD-CTT1298-Cy5 (7) in 81% yield.

¹H NMR (500 MHz, DMSO) δ 8.39-8.20 (m, Cy5 H), 8.14-7.05 (m, D-internal amide H, DBCO H, and Cy5 H), 6.59-6.49 (m, Cy5 H), 6.33-6.19 (m, Cy5 H), 5.86-5.73 (m, Cy5 H), 5.45-5.32 (m, Cy5 H), 4.94-4.47 (m, D-OH and ligand H), 4.37-3.84 (D-ester-CH₂ and ligand H), 3.21-2.97 (m, D-CH₂ and ligand H), 2.84-2.59 (m, D-CH₂), 2.37-1.99 (m, D-CH₂ and ligand H), 1.80-1.61 (m, ligand H and Cy5 H), 1.60-1.43 (m, linker —CH₂ and ligand H), 1.41-1.10 (m, linker —CH₂ Cy5 H, and ligand H).

Synthesis of compound 10: To a stirred solution of 4-amino-phenol 9 (874 mg, 8.0 mmol, 1.2 eq) was added sodium hydride (60% dispersion in mineral oil, 400 mg, 10.0 mmol, 1.5 eq) in dry N,N-dimethylformamide (10 mL) at 0° C. After 30 minutes, 7-benzyloxy-4-chloro-6-methoxy-quinoline 8 (2.0 g, 6.67 mmol, 1.0 eq) was added and reaction mixture was stirred at 80° C. for 16 h. The progress of the reaction was monitored with TLC. Crude reaction mixture was diluted with water and extracted with ethyl acetate (50 mL×4). The combined organic layer was dried over Na₂SO₄, filtered, and the solvent was evaporated. The final product was purified with silica gel chromatography (0-5% MeOH-DCM) to yield 2.2 g (Yield=92%) of 10 as yellow solid.

¹H NMR (500 MHz, DMSO-d₆) δ 8.42 (d, J=5.2 Hz, 1H), 7.49-7.57 (m, 3H), 7.47 (s, 1H), 7.41-7.46 (m, 2H), 7.34-7.41 (m, 1H), 6.89-6.97 (m, 2H), 6.63-6.71 (m, 2H), 6.37 (d, J=5.3 Hz, 1H), 5.30 (s, 2H), 5.17 (s, —NH₂). ¹³C NMR (125 MHz, DMSO-d₆) δ 161.4, 151.7, 149.7, 149.3, 147.1, 146.5, 143.8, 137.0, 128.9, 128.4, 128.4, 122.2, 115.5, 115.3, 109.6, 102.7, 99.8, 70.2, 56.1.

Synthesis of compound 12: Compounds 10 (2.0 g, 5.37 mmol, 1.0 eq) and 11 (1.8 g, 8.05 mmol, 1.5 eq) were dissolved in dry DMF (8 mL). DIPEA (2.0 mL, 10.74 mmol, 2.0 eq) and HATU (3.0 g, 8.05 mmol, 1.5 eq) were added to the solution, and the mixture was stirred overnight at room temperature. EtOAc (100 mL) was added, washed with water and brine, dried over Na₂SO₄. After filtration and condensation, the residue was purified by silica gel chromatography (0-100% EtOAc-Hexane) to yield 2.8 g (Yield=90%) of compound 12 as brown solid.

¹H NMR (500 MHz, DMSO-d₆) δ 10.04 (s, —NH), 10.27 (s, —NH), 8.65 (d, J=6.0 Hz, 1H), 7.82 (d, J=9.0 Hz, 2H), 7.62-7.69 (m, 2H), 7.51-7.58 (m, 4H), 7.45 (t, J=7.5 Hz, 2H), 7.39 (t, J=7.3 Hz, 1H), 7.31 (d, J=9.0 Hz, 2H), 7.16 (t, J=8.9 Hz, 2H), 6.66 (d, J=6.0 Hz, 1H), 5.36 (s, 2H), 4.00 (s, 3H), 1.47-1.54 (m, 4H). ¹³C NMR (125 MHz, DMSO-d₆) δ 168.6, 168.6, 162.1, 158.7 (d, J=238.7 Hz), 152.9, 150.4, 149.4, 147.7, 137.3, 136.6, 135.6 (d, J=2.6 Hz), 129.0, 128.6, 128.5, 122.8 (d, J=7.6 Hz), 122.6, 121.7, 115.6 (d, J=22 Hz), 115.4, 107.3, 103.5, 100.1, 70.6, 56.4, 32.1, 15.8.

Synthesis of compound 13: The compound 12 (1.5 g, 2.59 mmol) was dissolved in 6 mL of TFA and mixture was stirred at 60° C. for 30 minutes and concentrated. The mixture was basified to pH 7 with saturated aqueous NaHCO₃ and extracted with ethyl acetate (50 mL×3). The combined organic extracts were washed with brine and dried over anhydrous Na₂SO₄. After filtration, the filtrate was concentrated to afford a residue, which was purified by silica gel chromatography (0-10% MeOH-DCM) to yield 1.1 g (Yield=87%) of 13 as yellow solid.

¹H NMR (500 MHz, DMSO-d₆) δ 10.19 (s, —NH), 10.16 (s, —OH), 10.07 (s, —NH), 8.40 (d, J=5.2 Hz, 1H), 7.75 (d, J=9.0 Hz, 2H), 7.59-7.68 (m, 2H), 7.49 (s, 1H), 7.28 (s, 1H), 7.19-7.25 (m, 2H), 7.15 (t, J=8.9 Hz, 2H), 6.36 (d, J=5.2 Hz, 1H), 3.94 (s, 3H), 1.48 (s, 4H). ¹³C NMR (125 MHz, DMSO-d₆) 8 168.6, 168.6, 160.4, 158.7 (d, J=239 Hz), 151.2, 150.00, 149.5, 149.1, 147.0, 136.7, 135.6 (d, J=2.7 Hz), 122.9 (d, J=7.8 Hz), 122.6, 121.6, 115.5 (d, J=23 Hz), 115.0, 111.2, 102.8, 99.7, 79.6, 56.1, 32.0, 15.8. LCMS (ESI) calcd [M+H]⁺ for C₂₇H₂₂FN₃O₅: 488.1622, found 488.1032.

Synthesis of compound 15: To a mixture of dibenzocyclooctyne acid 14 (150 mg, 0.49 mmol, 1.2 eq) and compound 13 (200 mg, 0.41 mmol, 1.0 eq) in 10 mL of dry DCM was added EDC.HCl (118 mg, 0.61 mmol, 1.5 eq), DIPEA (0.1 mL, 0.61 mmol, 1.5 eq) and DMAP (5.0 mg, 0.04 mmol, 0.1 eq). The reaction mixture was stirred for 1 h at room temperature. Progress was monitored with TLC analysis. EtOAc (50 mL) was added, washed with water and brine, dried over Na₂SO₄. After filtration and condensation, the residue was purified by silica gel chromatography (0-10% MeOH-DCM) to yield 180 mg (Yield=57%) of Cabo-DBCO (15) as white solid.

¹H NMR (500 MHz, DMSO-d₆) δ 10.26 (s, —NH), 10.12 (s, —NH), 8.60 (d, J=5.1 Hz, 1H), 7.83 (d, J=8.7 Hz, 2H), 7.64-7.79 (m, 5H), 7.50-7.62 (m, 4H), 7.46 (t, J=7.4 Hz, 1H), 7.39 (t, J=7.5 Hz, 1H), 7.25-7.33 (m, 3H), 7.20 (t, J=8.8 Hz, 2H), 6.58 (d, J=5.1 Hz, 1H), 5.14 (d, J=14.1 Hz, 1H), 3.88 (s, 3H), 3.73 (d, J=14.0 Hz, 1H), 2.65-2.91 (m, 3H), 1.98-2.10 (m, 1H), 1.53 (s, 4H). ¹³C NMR (125 MHz, DMSO-d₆) δ 170.8, 170.7, 168.6, 168.5, 160.7, 158.7 (d, J=238.6 Hz), 151.7, 150.6, 150.1, 149.6, 148.7, 145.2, 143.6, 137.1, 135.6 (d, J=2.7 Hz), 132.8, 130.1, 129.4, 128.8, 128.5, 128.2, 127.3, 125.6, 122.9, 122.8 (d, J=7.8 Hz), 122.6, 122.1, 122.0, 121.7, 119.7, 115.5 (d, J=23 Hz), 114.8, 108.4, 104.6, 100.9, 56.6, 55.5, 55.4, 32.0, 29.8, 29.3, 15.8. LCMS (ESI) calcd [M+H]⁺ for C₄₆H₃₅FN₄O₇: 774.2568, found 775.1006.

Synthesis of compound 16: PAMAM-G4-OH (PD) dendrimer 4 (1.0 g, 1.0 eq, 0.07 mmol) was dissolved in Anhy. DMF (5 mL). Azido hexanoic Acid (154 mg, 14.0 eq, 0.98 mmol) was dissolved in Anhy. DMF (5 mL) and was activated by adding EDC.HCl (268.5 mg, 20.0 eq, 1.4 mmol) and stirred for 15 minutes. It was then added dropwise to the G4-PAMAM dendrimer solution under continuous stirring followed by addition of DMAP (77 mg, 9.0 eq, 0.63 mmol) and the reaction mixture was stirred for 24 hours at room temperature. The reaction completion was confirmed by the shift in the retention time of the chromatogram in HPLC. The dialysis was performed using a 1 kDa dialysis membrane in deionized water for 24 h. The aqueous solution was lyophilized to afford PD-azide (16) in 86% yield.

¹H NMR (500 MHz, DMSO) δ 8.15-7.68 (m, D-internal amide H), 4.81-4.61 (m, D-OH), 4.05-3.95 (m, D-ester-CH₂), 3.50-3.23 (m, D-CH₂), 3.18-2.98 (m, D-CH₂), 2.92-2.85 (D-CH₂), 2.78-2.55 (m, D-CH₂), 2.46-2.36 (m, D-CH₂), 2.35-1.99 (m, D-CH₂), 1.58-1.46 (linker —CH₂), 1.36-1.25 (linker —CH₂).

Synthesis of compound 17: To a solution of PD-azide 16 (20 mg, 0.0013 mmol, 1.0 eq) in DMF (100 μL) in 2 mL vial, was added solution of compound 15 (7.8 mg, 0.010 mmol, 8.0 eq) dissolved in DMF (100 μL). The reaction mixture was stirred at RT for 3 h. The reaction completion was confirmed by the shift in the retention time of the chromatogram in HPLC. Upon completion, the compound was purified with TFF 3 kDa dialysis membrane. The product was lyophilized to afford PD-Cabo (17) in 88% yield.

White fluffy Solid, ¹H NMR (500 MHz, DMSO) δ 10.14 (s, Cabo-amide H), 9.99 (s, Cabo-amide H), 8.46 (s, Cabo-Ar H), 8.06-6.96 (m, D-internal amide H, and Ar H), 6.45 (s, Cabo-Ar H), 6.0-5.73 (m, DBCO—CH₂), 4.66 (bs, D-OH), 4.53-4.08 (m, linker and Cabo H), 3.97-3.73 (m, D-ester-CH₂ and Cabo DBCO H), 3.85-3.31 (m, D-CH₂ and Cabo H), 3.11-2.91 (m, D-CH₂), 2.71-2.52 (m, D-CH₂ and Cabo H), 2.40-2.27 (m, D-CH₂ and Cabo H), 2.26-2.02 (m, D-CH₂ and Cabo H), 1.52-1.10 (m, D-linker H and Cabo H).

¹³C NMR (125 MHz, DMSO-d₆) δ 173.2, 172.0, 171.7, 170.8, 170.3, 168.6, 160.7, 159.6, 157.7, 150.7, 150.0, 149.6, 145.2, 144.2, 143.7, 142.6, 137.1, 135.6, 134.3, 131.2, 130.3, 122.9, 122.8, 122.6, 122.0, 121.7, 119.7, 115.5, 115.4, 115.0, 104.6, 100.9, 62.9, 60.3, 56.6, 52.6, 50.9, 50.0, 41.8, 40.5, 38.0, 37.3, 33.6, 33.6, 32.0, 29.3, 29.1, 28.4, 26.0, 25.4, 24.3, 24.1, 15.8.

Synthesis of compound 18: To a solution of PD-Cabo 17 (20 mg, 0.0009 mmol, 1.0 eq) in deionised water (100 μL) in a 2 mL glass vial was added solution of compound 3 (2.8 mg, 0.0028 mmol, 3.0 eq) dissolved in DI water (50 μL). The completion of reaction was tracked using HPLC. Upon completion, the product was lyophilized to afford PD-CTT1298-Cabo (18) in 91% yield.

White fluffy Solid, ¹H NMR (500 MHz, DMSO-d₆) δ 10.2-10.0 (m, Cabo-amide H), 8.55-7.05 (m, D-internal amide H and Cabo and DBCO Ar H), 6.51 (s, Cabo-Ar H) 6.06-5.73 (DBCO—CH₂), 5.16-4.59 (m, D-OH, ligand H), 4.53-3.69 (ligand H, linker H, and D-ester-CH₂), 3.41-2.79 (m, D-CH₂, ligand H, and Cabo H), 2.76-2.55 (m, D-CH₂ and Cabo H), 2.47-2.33 (m, D-CH₂, Cabo H, and ligand H), 2.32-2.01 (m, ligand H), 1.63-0.78 (m, D-linker H and Cabo H). ¹³C NMR (125 MHz, DMSO-d₆) δ 0.84-1.48 (m, 107H), 1.48-1.71 (m, 75H), 2.26 (s, 250H), 2.40-2.54 (m, 112H), 2.69 (s, 238H), 3.02-3.21 (m, 193H), 3.25-3.36 (m, 50H), 3.36-3.54 (m, 167H), 3.84-4.09 (m, 57H), 4.14-4.67 (m, 59H), 4.70-5.32 (m, 60H), 5.86 (d, J=39.1 Hz, 6H), 6.02 (dd, J=53.3, 17.4 Hz, 16H), 6.58 (s, 8H), 7.20 (t, 29H), 7.25-7.53 (m, 62H), 7.53-7.75 (m, 63H), 7.74-7.99 (m, 49H), 7.99-8.47 (m, 89H), 8.56-8.62 (m, 8H), 10.12-10.35 (m, 16H). ³¹P NMR (202 MHZ, D₂O) δ 7.34. MALDI-TOF: Theoretical: 25.5 kDa; obtained: 24.1 kDa.

### Drug Release Studies

In vitro drug release studies were conducted under plasma conditions (phosphate-buffered saline, PBS, pH 7.4) and intra-tumoral conditions (citrate buffer, pH 5.5, containing esterase). PD-CTT1298-Cabo conjugate was dissolved at a concentration of 1 mg/mL in each respective buffer and underwent incubation at 37° C. with continuous shaking to replicate physiological conditions. At specific time intervals, samples were withdrawn, promptly quenched with an equivalent volume of methanol, and subsequently stored at −20° C. until further analysis. The released drug was then analyzed using HPLC, and the extent of drug release was determined by comparing it to the standard curve established for free drug (Cabo-OH) on the HPLC system.

### In Vitro Studies

Confocal microscopy: 5×10⁵ PC3-PIP PSMA (+) cells and PC3 PSMA (−) cells were added to glass microscopy slides and incubated overnight. Following incubation, the media was aspirated, and fresh serum free media was added. Cells were then treated with 50 μg/mL (2.5 μM) of the fluorescently labeled PD-CTT1298-Cy5 for 30 min. Following incubation at 37° C., the media was discarded, and the slides were washed 3× with ice cold PBS. The cells were then treated with 10 μg/mL (36 μM) DAPI at RT for 10 min. Following treatment of cells with DAPI, the supernatant was aspirated, the cells were washed 3× with ice cold PBS, and fixed with 4% formaldehyde at RT for 10 min. Finally, the cells were washed 3× with ice cold PBS and coverslips were mounted for visualization using an SP-8 Confocal Microscope equipped with a 63× lens.

Flow cytometry: To determine whether the PD-CTT1298-Cy5 dendrimer was selectively targeted to PSMA (+) cells, a quantitative cell uptake experiment was performed. 5×10⁵ PC3-PIP PSMA (+) cells and PC3 PSMA (−) cells were added to individual Eppendorf vials. Cells were then treated with 1 μg/mL (50 nM) of the fluorescently labeled PD-CTT1298-Cy5 dendrimer at various time points (T=0, 0.5, 1, and 2 H). Following incubation at 37° C., cells were centrifuged at 1200 RPM for 2 min and the supernatant was discarded. The samples were washed 3× with ice cold PBS, fixed with 4% formaldehyde at RT for 10 min. Following fixing the cells, the samples were centrifuged at 1200 RPM for 2 min, washed 3× with ice cold PBS and suspended in FACS Buffer for analysis via flow cytometry. Blank cells were run to set an appropriate gate and 10,000 events were collected per sample. The Mean Fluorescence Intensity (MFI) was acquired and normalized. For the blocking experiments, the same procedure was repeated except the cells were incubated with CTT 1057 (500 nM) for 30 min prior to the addition of PD-CTT1298-Cy5 dendrimer. For the dose response experiment, the cells were incubated with different concentrations (0.1 nM-50 nM) of the dendrimer for 1 H.

Cell viability: 2.5×10⁴ PC3-PIP PSMA (+) cells were allowed to adhere to a 96 well plate overnight. The following day, cell media was changed and the PD-CTT1298-Cabo conjugate, at the concentrations tested, was added along with cabozantinib (Cabo) and hydoxy-cabozantinib (Cabo-OH). The cells were allowed to incubate at 37°° C. for 72H. Following incubation, the luminescence of the viable cells was measured using the CellTiter-Glo® Luminescent Cell Viability assay according to manufacturer's instructions. Cell viability (%) was calculated using the luminescence values obtained from the controls used in the experiment. The experiment was performed in triplicate.

### Animal Studies and in Vivo Fluoresceine Imaging

The animal studies received prior approval from the Washington State University (WSU) IACUC and complied with IACUC recommendation. Male 5-week-old athymic nude mice were purchased from the Jackson Laboratory and housed in the animal research facility at WSU Spokane. The control (PSMA−) PC3 and PSMA overexpressing (PSMA+) PC3-PIP cell lines were cultured in RPMI medium containing 10% fetal bovine serum and 1% penicillin-streptomycin. 2×10⁶ PC-3 cells were mixed 1:1 with Matrigel® solubilized basement membrane matrix (BD Biosciences) and injected subcutaneously into the flank of the nude mice to allow tumor development. For in vivo fluorescence imaging, mice growing PSMA+ PC-3 PIP tumors at a size of 150-200 mm³ were randomly divided into two groups to receive intravenous injection of a blocking agent (100 μL injection volume) or not, 30 mins prior to imaging (n=3 for each group). Then all tumor-bearing mice were intravenously injected with PD-CTT1298-Cy5 at the dose of 20 mg/kg (100 μL injection volume) and subjected to whole-body fluorescence imaging at 1, 6, 24, and 48 H after injection using an IVIS™ SpectrumCT In Vivo Imaging System (PerkinElmer). At the endpoint, the mice were sacrificed and dissected to obtain tumors and vital organs for ex vivo fluorescence imaging and quantification. The fluorescence intensities in regions of interest (ROI) were calculated using the Indigo software equipped with the IVIS™ instrument. Whole blood was drawn from the heart and placed in a heparin-coated tube. Tissues were snap-frozen in liquid nitrogen and stored at −80° C.

### Ex Vivo Dendrimer Quantification via Fluorescence Spectroscopy

Ex vivo quantification of dendrimers was carried out by thawing the frozen organs (tumor, heart, lungs, liver, kidneys, spleen, and brain) gradually on ice and weighing them. Known amounts of tissues from each organ were taken, weighed, and homogenized with stainless steel beads in methanol at a concentration of 1 mL/100 mg of tissue using a tissue homogenizer. The homogenized samples were then centrifuged at 4° C., and the clear supernatant was transferred to Eppendorf® tubes and stored at −80° C. in dark. For fluorescence quantification, the thawed supernatants were centrifuged again, and fluorescence intensity of the supernatant was measured using a Horiba Fluoromax® spectrofluorophotometer. The fluorescence intensity for Cy5 (λex=645 nm, λem=662 nm) was determined. These fluorescence intensity values were converted to dendrimer concentrations using calibration curves of PD-CTT1298-Cy5 at different slit widths.

### PSMA IC₅₀ for PD-CTT1298

The routine determination of IC₅₀, as most recently described in our laboratory, for PD-CTT1298 was achieved using concentrations of 160, 80, 40, 20, and 10 nM. Results are presented as mean±standard error of the mean (SE).

### Statistical Analysis

The statistical analyses were performed using Student's 2-tailed t-test with unequal variances.

### Results and Discussion

### Synthesis of Fluorescently Labelled PD-CTT1298

By combining PSMA targeting through irreversible ligand (CTT1298) with a nanotechnology-based approach employing dendrimers, we have precisely engineered systemic PSMA-targeting dendrimers (PD-CTT1298) for enabling targeted intracellular delivery of potent chemotherapeutic agents to tumor cells. It is worth noting that while a few PSMA-targeted dendrimers have been assessed for targeting and drug delivery, these prior endeavours were based on cationic (positively charged) dendrimers. However, the toxicity concerns of cationic dendrimers due to their interactions with negatively charged cell membranes, has impeded their clinical translation. On the contrary, neutral PAMAM-G4-OH dendrimers (PD), which are non-cytotoxic, and biocompatible, have emerged as promising candidates for target-specific drug delivery applications. Moreover, PAMAM-G4-OH dendrimers exhibit excellent water solubility making them favourable nanocarriers for drug delivery applications. The molecular and structural confirmations of PAMAM dendrimers have been extensively studied in literature using molecular dynamics simulations, which suggest that the generation 4 PAMAM dendrimers, in the presence of a favorable solvent, like water, assume spherical shape, with branches stretched out and surface groups protruding outside. Water is a choice of solvent for drug delivery applications. Even though these molecular simulation studies were conducted on amine-terminating PAMAM dendrimers, we expect similar configuration for hydroxyl-terminating PAMAM dendrimers in water, where terminal amines are replaced with hydroxyl groups keeping the same backbone. We chose CTT1298 as the PSMA-targeting agent due to its high-affinity and irreversible binding to PSMA (IC₅₀ 19 nM), that translated to extensive internalization in PSMA (+) tumor cells. However, its highly charged nature due to the presence of multiple carboxylate groups leads to rapid renal clearance, that can be avoided via dendrimer conjugation by increasing its blood circulation time. Moreover, dendrimers also provide a platform for the attachment of high payloads of therapeutic molecules combing drug delivery with targeting. We opted to attach 3 molecules of CTT1298 on the periphery of dendrimers based on the following rationale. An analogue of CTT1298 known as TG97 was used to deliver the enzyme yeast cytosine deaminase (yCD) to PSMA-positive cells. In addition, three molecules of CTT54 (another CTT1298 analogue) were used to deliver Cy5-streptavidin to PSMA-positive cells. Both of these examples confirm that highly-potent small molecule ligands to PSMA can deliver large molecular cargo. Moreover, CTT1298 has several carboxylic acids and a phosphonic acid group. Attachment of large number of ligands may lead to a negatively charged dendrimer conjugate that may show non-specific uptake, as previously reported for negatively charged nanoparticles. To achieve PSMA specific targeting with minimal accumulation of dendrimer at off-target organs, we opted to attach only ˜3 targeting ligands. The conjugation of CTT1298 targeting ligand on the surface of PD was achieved employing highly efficient and robust Strain-Promoted Alkyne Azide Cycloaddition (SPAAC) reactions. The selectivity of SPAAC enables controlled reactions, precise ligand, or drug loading, minimizing unwanted byproducts, and its ability to operate under mild conditions, including physiological temperatures, ensures its applicability to a wide range of applications. Its versatility in conjugating different molecules, from ligands to drugs and imaging agents, underscores its utility in creating targeted drug delivery systems, imaging probes, and facilitating biomolecular labelling.

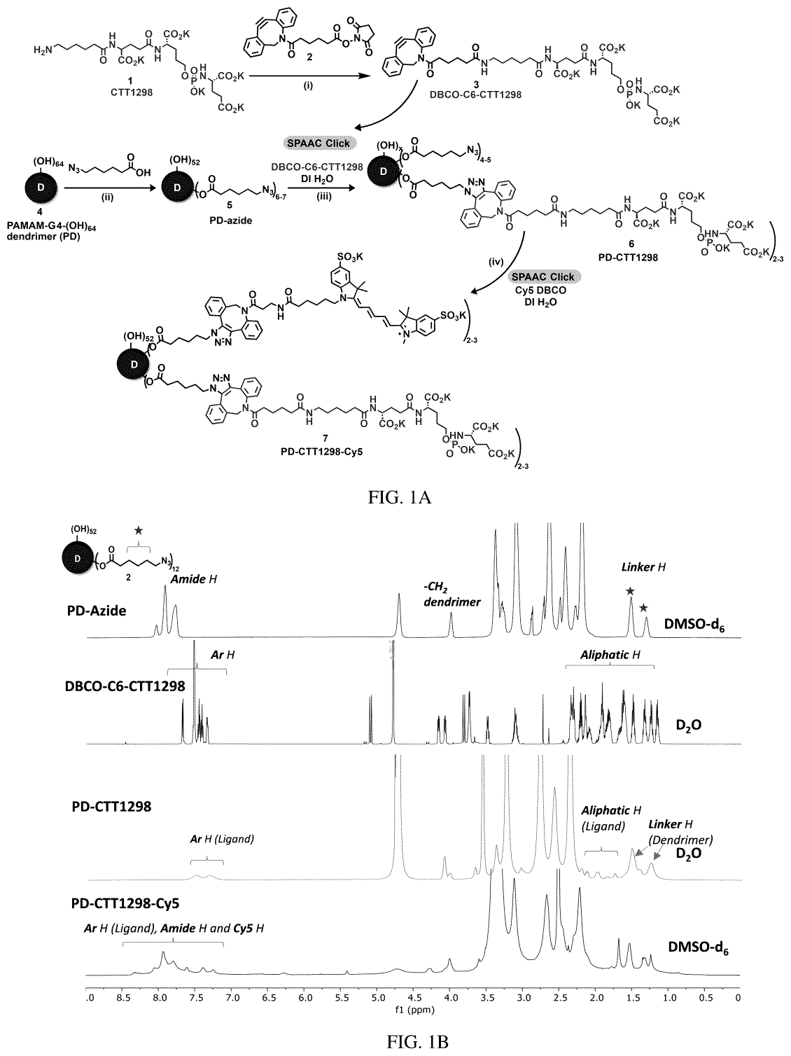

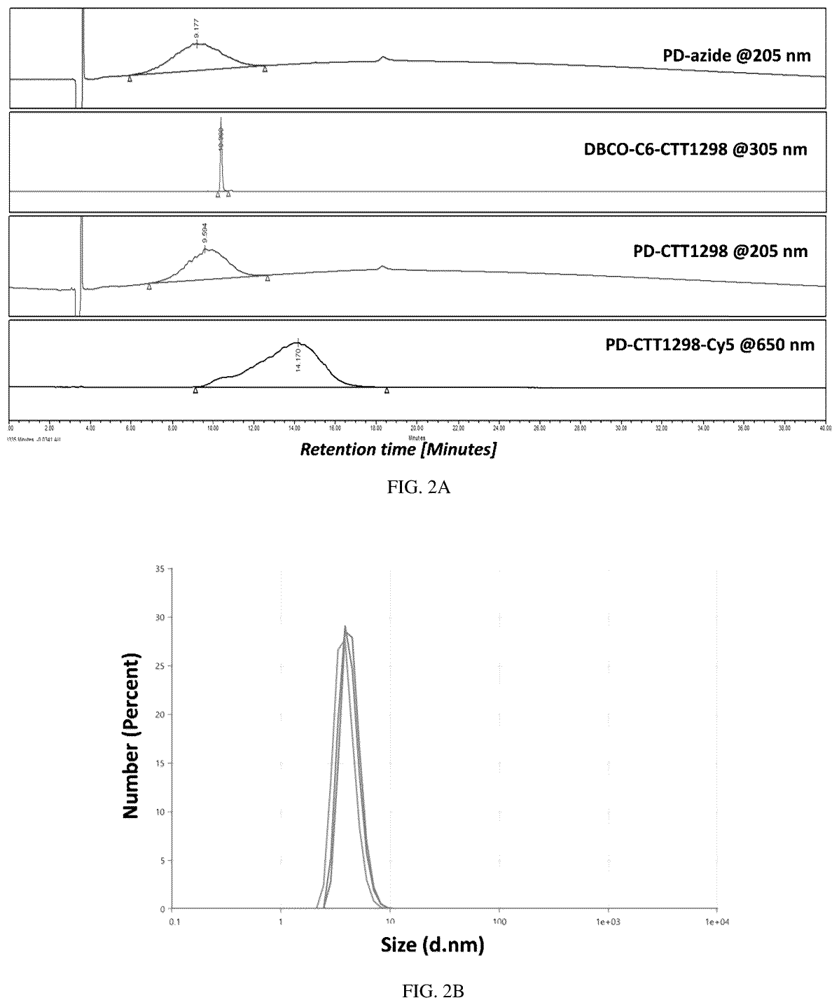

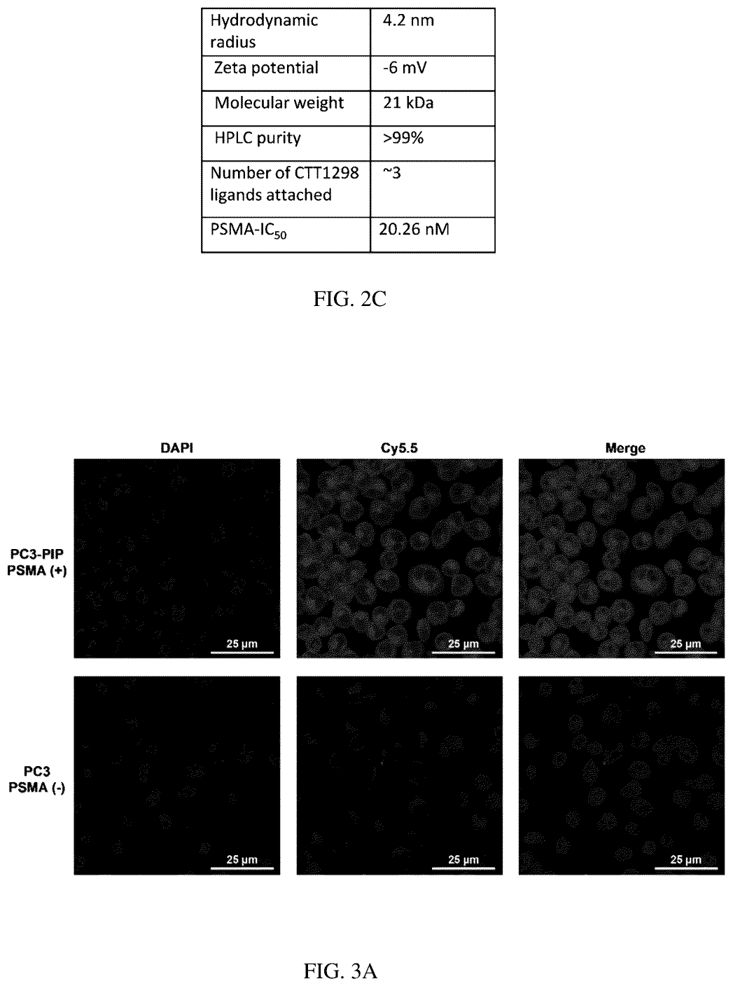

The synthesis of PD-CTT1298-Cy5 was initiated with the modification of CTT1298 (1) to bring dibenzocyclooctyne (DBCO) group to participate in SPAAC reaction (FIG. 1A). This was achieved by reacting compound 1 with DBCO-NHS ester (2) to obtain DBCO-C6-CTT1298 (3). The presence of DBCO protons in the aromatic region along with the ligand protons confirmed the product formation (FIG. 1B). Next, the hydroxyl groups on PD (4) were partially modified through a reaction with azido hexanoic acid via Steglich esterification (FIG. 1A), resulting in a partially azide-terminated dendrimer (5) with approximately six periphery azides, confirmed by the appearance of linker protons between δ 1-2 ppm and dendrimer-ester methylene protons at δ 4.7 ppm in ¹H NMR (FIG. 1B). Subsequently, the PD-Azide (5) and DBCO-C6-CTT1298 (3) were conjugated via SPAAC reaction in deionized (DI) water. SPAAC facilitated the conjugation of the ligand onto the dendrimer surface in the desired equivalents to yield PD-CTT1298 (6) with ˜3 ligand molecules attached, confirmed by the comparative integration of aromatic protons from the ligand between δ 7 and 8 ppm and linker protons from dendrimer in the aliphatic region (FIG. 1B). The HPLC chromatogram exhibited a shift in retention time, transitioning from 9.2 minutes for PD-azide to 9.6 minutes for PD-CTT1298 upon the conjugation of the PSMA targeting ligand (FIG. 2A). The HPLC purity level of PD-CTT1298 exceeded 99% (FIG. 2A). The size and zeta potential distribution of PD-CTT1298 was analyzed using dynamic light scattering (DLS). The hydrodynamic radius of PD-CTT1298 was found to be 4.2±0.2 nm and the zeta potential distribution was −6 mV (FIGS. 2B & 2C). To evaluate whether CTT1298 conjugated to the dendrimer retained activity as an inhibitor of PSMA, the PSMA-IC₅₀ was evaluated using our previously published procedure. The IC₅₀ of PD-CTT1298-Cy5 was still in nM range (20.26 nM), suggesting the retention of PSMA targeting ability upon dendrimer conjugation (FIG. 2C).

To further investigate the in vitro and in vivo PCa cell uptake and organ biodistribution of PD-CTT1298 via confocal and fluorescence spectroscopy, a near-infrared dye cyanine 5 (Cy5) was introduced at its surface. Subsequent SPAAC reaction of PD-CTT1298 with Cy5-DBCO yielded the final fluorescent dendrimer PD-CTT1298-Cy5 (7). Confirmation of Cy5 attachment was achieved through the observation of Cy5 protons in the ¹H NMR spectrum (FIG. 1B). Using the proton integration method, the calculation indicated the attachment of approximately two Cy5 molecules on the dendrimer surface (FIG. 1B). PD-CTT1298-Cy5 demonstrated a purity exceeding 98% in HPLC, with the chromatogram showing a significant shift in retention time from 9.6 to 14.2 minutes upon Cy5 conjugation (FIG. 2A). All the intermediates and final conjugates were characterized using NMR and Mass spectroscopy and the purity was analyzed using HPLC (data not shown).

### PD-CTT1298-Cy5 Demonstrates Selective Uptake in PSMA (+) PC3-PIP Cells via PSMA Mediated Internalization

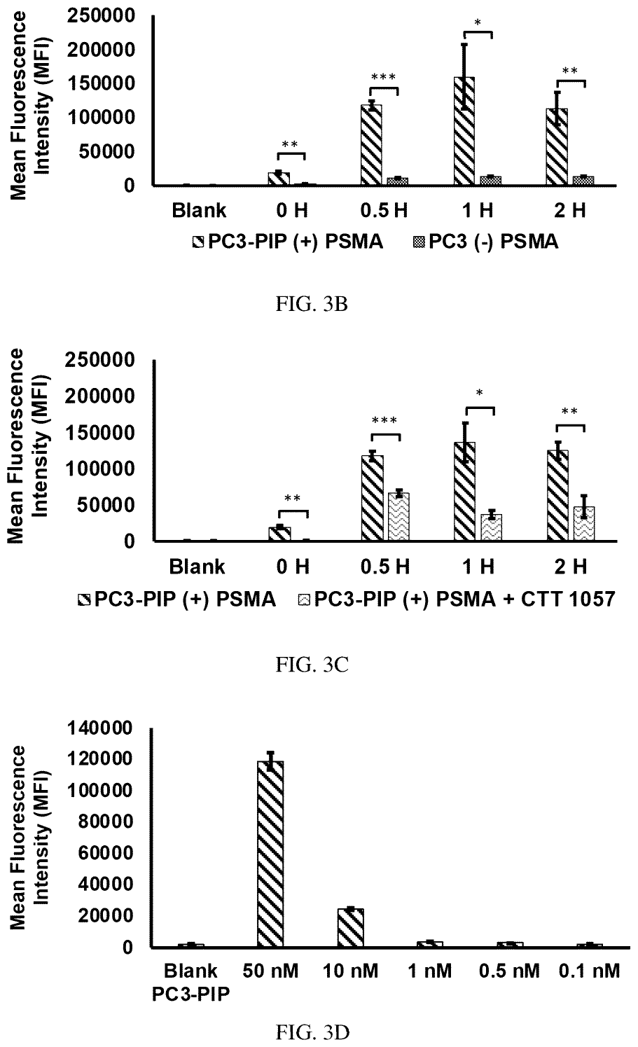

Next, to investigate the selective uptake of the PD-CTT1298-Cy5 dendrimer in PSMA (+) cells, cell uptake experiments were performed under various conditions (FIG. 3). We first analyzed the qualitative uptake of PD-CTT1298-Cy5 in PSMA (+) and PSMA (−) cells using confocal microscopy. The PSMA (+) PC3-PIP and PSMA (−) PC3 cells when incubated with 50 μg·mL⁻¹ (2.5 μM) of the PD-CTT1298-Cy5, revealed the selective uptake of PSMA-targeted PD-CTT1298-Cy5 in PSMA (+) cells (FIG. 3A). A negligible uptake was observed in PSMA (−) PC3 cells. We next evaluated the quantitative uptake and mechanism of uptake of dendrimers using flow cytometry. At 1 μg/mL (50 nM) concentration, selective uptake was observed in PSMA (+) PC3-PIP cells over 2 H (FIG. 3B). As expected, in PC3-PIP cells, the Mean Fluorescence Intensity (MFI) increased significantly over the time points due to their high PSMA expression. A significant change in MFI (˜150-fold increase) was observed in as little as 30 minutes (FIG. 3B) when compared to the 0 H and blank samples. Minimal uptake of the PD-CTT1298-Cy5 dendrimer was observed in PC3 cells, which express little to no PSMA. The uptake was significantly more in PSMA (+) cells compared to PSMA (−) cells at all time-points. The histograms obtained also showed a 2-log shift in the PSMA (+) cells (data not shown) compared to PSMA (−) cells (data not shown), which was anticipated based of their PSMA expression. The results clearly demonstrate that the uptake of PD-CTT1298-Cy5 corelates with the PSMA expression on the cells, suggesting that the nanoplatform could serve as an effective means for targeted drug delivery to PSMA-positive prostate cancer cells.

To study any non-specific uptake of the PD-CTT1298-Cy5 in PSMA (+) cells and to further confirm the mechanism of uptake via PSMA receptors, a blocking experiment was performed at different time points in the presence of a potent irreversible PSMA inhibitor (CTT 1057). In the blocking experiment, a drastic decrease in the uptake of the PD-CTT1298-Cy5 was observed when PC3-PIP cells were incubated with a PSMA irreversible inhibitor with nanomolar affinity, CTT 1057 (FIG. 3C). A significant decrease in MFI was observed at all time points when the cells were incubated with CTT 1057. A dose response study was performed at 1H with different concentrations of PD-CTT1298-Cy5 (FIG. 3D). The selective uptake was dose dependent with the highest concentration tested (50 nM) resulting in the highest MFI. All together, these in vitro results clearly suggested that the PD-CTT1298-Cy5 was selectively targeted and taken up by cancer cells that expressed high levels of PSMA and internalized rapidly intracellularly following binding to PSMA.

### Qualitative and Quantitative Tumor and Organ Biodistribution of Systemically Administered PD-CTT1298-Cy5 in a PCa Tumor Xenograft Mouse Model

We further explored the in vivo tumor-targeting potential and biodistribution of CTT-1298-Cy5 in a human PC3 (PSMA−) and PC3-PIP (PSMA +) tumor xenograft model. To study the mechanism of uptake, the tumor uptake and biodistribution was also studied in PC3-PIP (PSMA +) tumor xenograft model in the presence of a blocking agent, CTT1057. Upon intravenous administration of PD-CTT1298-Cy5 dendrimers in PSMA (−) and PSMA (+) tumor-bearing mice, Cy5.5 fluorescence signals arising from the tumors and mouse vital organs were measured and compared at different time points by the in vivo imaging system (IVIS) imaging. The fluorescence signal accumulated in the mouse organs in general decreased more rapidly with time than that in tumors across all three groups and appeared to be invisible starting 24 H post-injection. This clearly suggests the rapid clearance of dendrimers from off-target organs and tissues, which is highly desired for targeted delivery of potent chemotherapeutic agents where systemic side-effects are a major concern. Furthermore, while most other nanoparticles when systemically administered show unwanted accumulation of up to 80% in the liver, the PD-CTT1298-Cy5 takes advantage of its small size (˜4 nm) in the range of renal filtration and clears intact through kidneys as demonstrated earlier for PD nanoplatform.

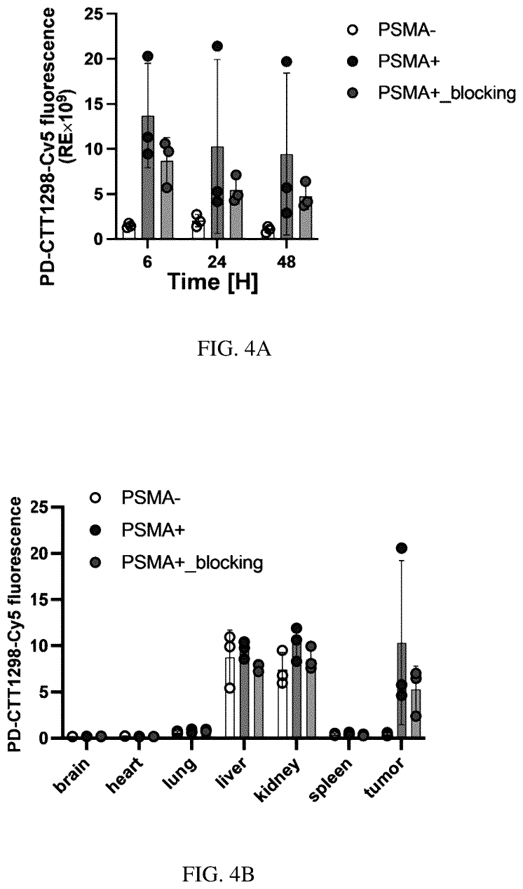

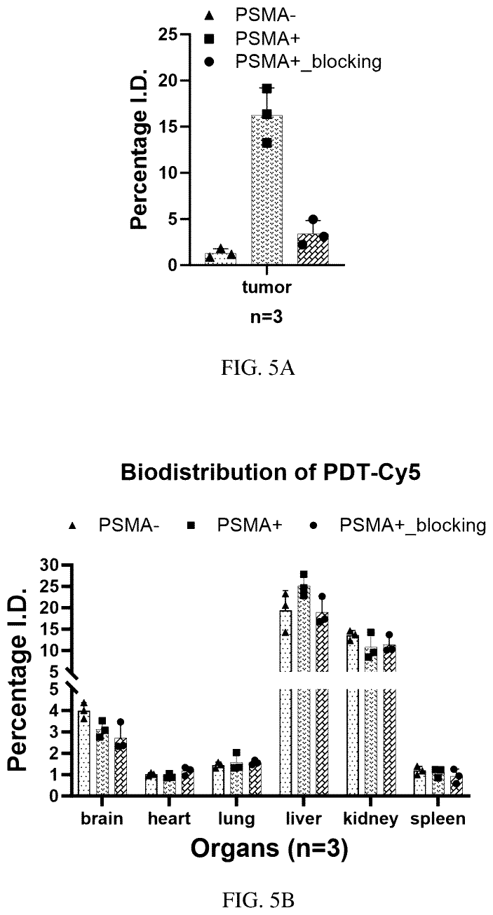

PD-CTT1298-Cy5 is selectively targeted to PSMA (+) PC3-PIP tumor and did not show accumulation in the mice with PSMA (−) PC3 tumors. The lack of discernible fluorescence signal since 6 H post-injection suggested rapid clearance from PSMA (−) tumors. However, in PSMA (+) tumor, PD-CTT1298-Cy5 demonstrated stable and intense fluorescence signal starting 6 H post-injection which was retained up to 48 H and was dimmed out by prior uptake of a blocking agent, CTT1057, further confirming the mechanism of uptake through PSMA receptors (FIG. 4B). In line with the in vivo observations, the endpoint ex vivo fluorescence imaging further indicated a high level of fluorescence signal in PSMA (+) tumors, which tended to be lower in PSMA (+) tumors with pre-treatment of a blocking agent and absent in PSMA (−) tumors (FIG. 4B). This was further confirmed by the quantitative uptake of PD-CTT1298-Cy5 in tumor tissues from all three groups using fluorescence spectroscopy. While the dendrimer uptake in PSMA (+) tumor group was ˜15% of the injected dose (ID), it decreased to ˜4% upon PSMA blocking, and was <2% in PSMA (−) tumor group (FIG. 5A), correlating to the ex-vivo fluorescence imaging by IVIS.

A significant challenge in the clinical application of nanomedicine-based therapeutics is their potential undesired accumulation in off-target organs. We next examined the ex vivo qualitative and quantitative distribution of PD-CTT1298-Cy5 in key organs, including the heart, lungs, liver, spleen, and kidneys at 48 H via IVIS and fluorescence spectroscopy (FIGS. 4B and 5B). The PD-CTT1298-Cy5 showed minimum accumulation in the vital organs including brain, heart, lungs, and spleen. The PD-CTT1298-Cy5 levels in the heart, lungs, spleen, and brain were found to be less than 5% ID in all groups, indicating the rapid clearance of the dendrimer from these organs by the 48 H time point (FIG. 5B). This was consistent with the ex vivo imaging of these organs (FIG. 4B). The ex vivo imaging showed some accumulation in liver and kidneys (FIG. 4B). In line with ex vivo imaging results, the tissue quantification suggested ˜20% and ˜10% ID of PD-CTT1298-Cy5 in liver and kidneys respectively (FIG. 5B). Although there is some non-specific liver uptake, the fluorescence signal in kidneys is rather expected due to the renal clearance mechanism of PD-CTT1298-Cy5.

To summarize these biodistribution results, 1) the uptake of PD-CTT1298-Cy5 was significantly higher in the tumor of the PSMA (+) group compared to both the PSMA (+) plus blocking and PSMA (−) groups, suggesting the uptake of PD-CTT1298-Cy5 in the tumor through PSMA mediated targeting; 2) PD-CTT1298-Cy5 cleared rapidly from other organs, indicating the targeted delivery of PD-CTT1298-Cy5 to the prostate tumor, and 3) there was a significant presence of PD-CTT1298-Cy5 in the tumor region of the PSMA (+) group at 48 H post-administration, suggesting the long-lasting sustained retention of PD-CTT1298-Cy5 in the prostate tumor regions. Collectively, these results suggested that PD-CTT1298-Cy5 dendrimers target PSMA (+) prostate tumors with preferential specificity making it a potential platform for the targeted delivery of potent chemotherapeutic agents to prostate cancer with positive PSMA expression.

### Synthesis and Characterization of PSMA-Targeted Dendrimer Cabozantinib Conjugate

To further investigate if PD-CTT1298-Cy5 can be utilized to deliver chemotherapeutic agents for the treatment of PCa, we developed its drug conjugate with cabozantinib (Cabo). Cabo is a multi-tyrosine kinase inhibitor and is approved as a single agent for renal cell and hepatocellular carcinoma in the USA and Europe. Unfortunately, its PCa clinical trial (Phase III) was terminated due to significant negative effects at tolerated doses. However, in preclinical testing, Cabo exhibited significant inhibition of advanced PCa tumor progression. Cabo in combination with immunotherapy is now a standard treatment in metastatic renal cancer, and its efficacy is being tested in PCa. Therefore, we hypothesized that the PD-CTT1298 mediated intracellular delivery of Cabo may enhance its efficacy and reduce the negative side effects.

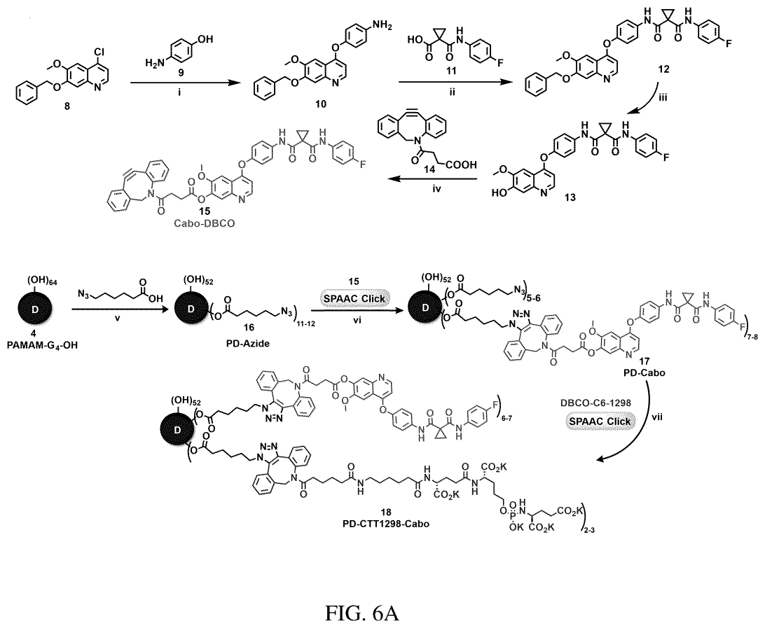

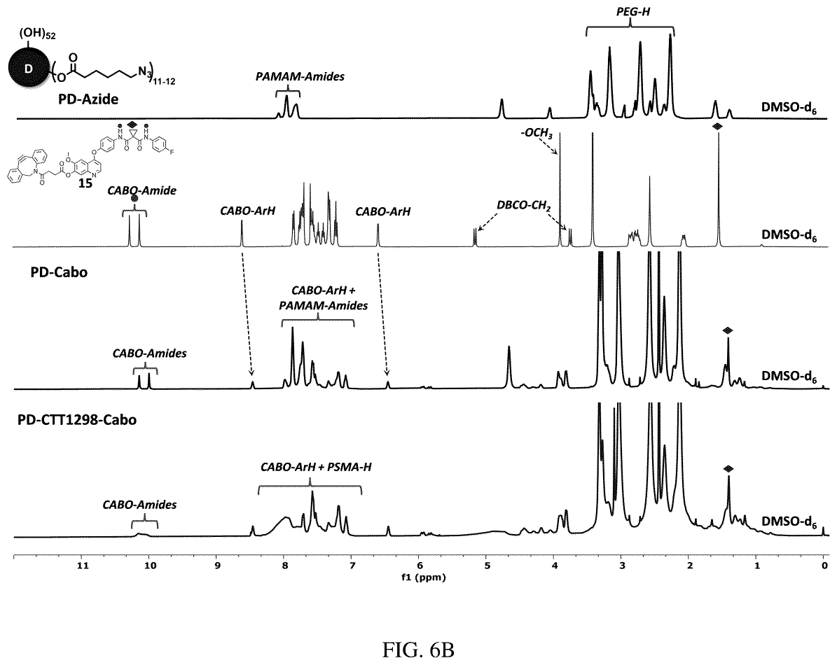

The synthesis of PSMA-targeted dendrimer cabozantinib conjugate (PD-CTT1298-Cabo) began with the synthesis of DBCO modified Cabo (Cabo-DBCO) (FIG. 6A). The DBCO modification was carried out at position 7, on the solvent exposed site of Cabo. It has been previously reported that the modification at position 7 of Cabo, did not alter the activity towards c-Met. To synthesize Cabo-DBCO, we first synthesized 7-demethylated cabozantinib intermediate (Cabo-OH; 13) followed a previously published protocol, with slight modifications. The reaction initiated by treating 7-(benzyloxy)-4-chloro-6-methoxyquinoline (8) with 4-aminophenol (9) in the presence of NaH in DMF, resulting in the formation of 4-((7-(benzyloxy)-6-methoxyquinolin-4-yl)oxy)aniline (10) in 92% yield. The desired product formation was confirmed through the observation of a proton NMR signal of —NH₂ at δ 5.1 ppm (data not shown). Subsequently, the condensation of the amino intermediate (10) with 1-((4-fluorophenyl) carbamoyl) cyclopropane-1-carboxylic acid (11) was carried out using HATU and DIEPA in dichloromethane, yielding compound 12. The structure of coupling product was validated by the appearance of two amide (—NH) proton peaks at δ 10.05 and 10.23 ppm, along with a cyclopropane ring proton singlet at δ 1.48 ppm (data not shown). Finally, the removal of the benzyl protecting group at the 7^{th} position was accomplished through treatment with trifluoroacetic acid at 60° C. for 30 minutes, resulting in the formation of the 7-demethylated cabozantinib intermediate (13) in 87% yield. The completion of the deprotection was evident from the ¹H NMR, which showed the disappearance of benzyl (—OBn) proton signals in the aromatic region and its corresponding methylene (—OCH₂) protons in the aliphatic region. Additionally, a hydroxyl peak at δ 10.16 ppm (data not shown) further confirmed the structure of the intermediate. The mass spectra analysis also provided the confirmation of the successful formation of intermediate 13 (data not shown). Further, the synthesis of compound 18, Cabo-DBCO, was achieved through the coupling of DBCO acid with Cabo-OH (13) in the presence of EDC-DMAP. The confirmation of the desired structure was established by the disappearance of the hydroxyl protons of Cabo-OH at δ 10.16 ppm. Simultaneously, the emergence of distinctive peaks corresponding to the DBCO ring's —CH₂ protons at δ 3.73 ppm (d, J=14.0 Hz, 1H) and 5.14 ppm (d, J=14.1 Hz, 1H), along with the DBCO aromatic protons in the ¹H NMR spectrum, validated the successful synthesis of Cabo-DBCO (FIG. 6B).

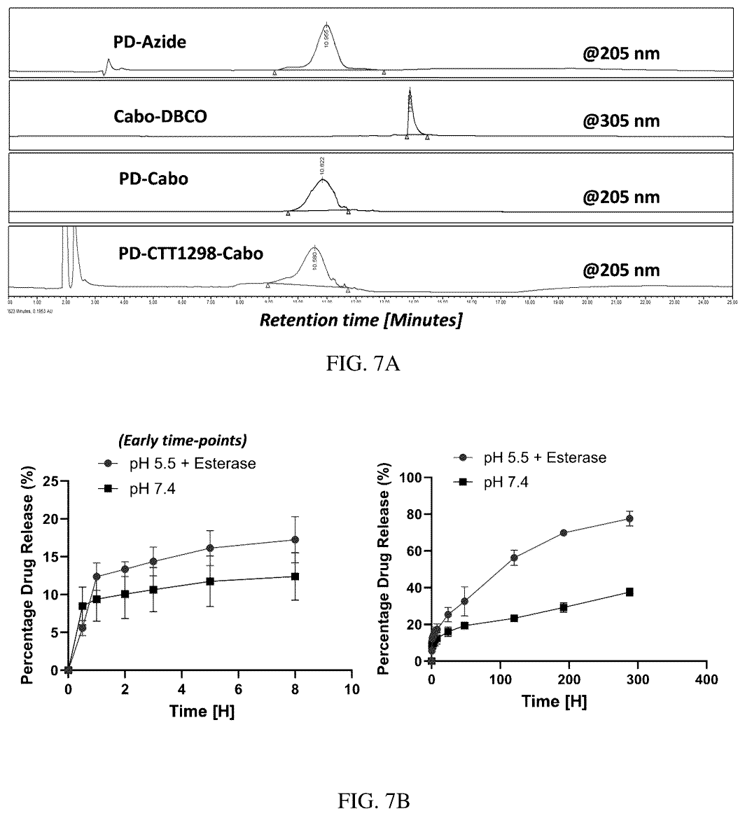

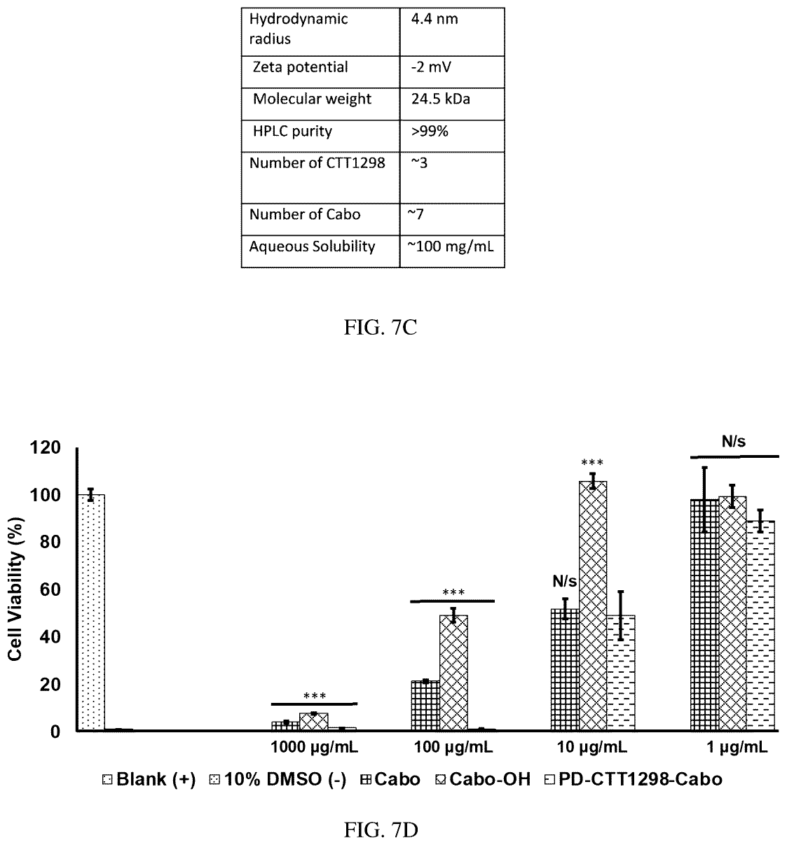

On the other hand, PD was modified to bring ˜12 azide groups to obtain PD-azide (16), which was reacted with Cabo-DBCO (15) using SPAAC reaction in DMF to obtain PD-Cabo conjugate 17 (FIG. 6A). The SPAAC reaction exhibited rapid kinetics and enabled the conjugation of an exact equivalent of Cabo without the need for any additional reagents. The reaction progress was monitored by HPLC, revealing a distinct shift in the chromatogram (10.95 to 10.82 min) as Cabo was successfully linked to the dendrimer surface (FIG. 7A). After dialysis purification and freeze-drying, the PD-Cabo was obtained in 88% yield. The successful conjugation of Cabo onto the dendrimer was verified using ¹H NMR, where the emergence of characteristic Cabo peaks was observed alongside the dendrimer protons. ¹H NMR confirmed the attachment of approximately 8 drug molecules per dendrimer corresponding to ˜16 weight percent drug loading. (FIG. 6A). Our previous work on PD platform for targeted drug delivery applications demonstrates that the dendrimer platform retains its targeting capabilities and can successfully deliver the drugs to targeted intracellular locations when the drug loading is in the limit of 20 weight percent. The purity of PD-Cabo conjugate was ˜99% by HPLC (FIG. 7A). After the attachment of Cabo to the dendrimer surface, the conjugation of the PSMA ligand, CTT1298, onto the dendrimer was carried through SPAAC chemistry. The Compound 17 was treated with the DBCO-C6-CTT1298 in DI water at room temperature for 3 hr, resulting in the synthesis of PD-CTT1298-Cabo (18). The HPLC chromatogram exhibited a shift from 10.82 to 10.58 min (FIG. 7A), and the appearance of additional protons in the ¹H NMR spectrum indicated the successful incorporation of the ligand onto the dendrimer (FIG. 6B). Furthermore, a signal at δ 7.34 ppm in the ³¹P NMR spectrum validated the presence of the PSMA ligand on the dendrimer surface (data not shown). Utilizing the proton integration method, the number of attached CTT1298 molecules on the dendrimer surface was calculated, suggesting the attachment of approximately three molecules of CTT1298. The HPLC analysis indicated a purity of PD-CTT1298-Cabo exceeding 98% (FIG. 7A). All the intermediates and final conjugates were characterized using NMR and mass spectroscopy, and HPLC techniques (data not shown). The physicochemical properties of PD-CTT1298-Cabo are presented in FIG. 7C. While both Cabo and Cabo-OH demonstrate poor aqueous solubility, dendrimer conjugation significantly improves the water solubility. The aqueous solubility of PD-CTT1298-Cabo is ˜100 mg/mL which translates to ˜16 mg/mL for Cabo-OH. The hydrodynamic radius of PD-CTT1298-Cabo is 4.40±0.07 nm and zeta potential distribution is −2.0±0.7 mV as analysed by the DLS (FIG. 7C). Cabo conjugation did not have much effect on the size and zeta potential of the PD-CTT1298 dendrimer, which was important to maintain the targeting potential of dendrimer intact. We further assessed the comparative c-Met inhibition activities (IC₅₀) of PD-CTT1298-Cabo versus Cabo and Cabo-OH. The PD-CTT1298-Cabo conjugate exhibited a nanomolar c-Met inhibitory activity (IC₅₀: 0.423 nM) that was better than both Cabo-OH (IC₅₀: 26.3 nM) and Cabo (IC₅₀: 1.3 nM) which could be due to the multivalency effect of dendrimers. These data confirm that Cabo is still active when conjugated in this manner.

### In Vitro Drug Release Study from PD-CTT1298-Cabo Conjugate Under Physiological Conditions

Next, we carried out an in vitro drug release study from the conjugate, examining conditions that mimic both the extracellular environment (physiological pH, PBS buffer at pH 7.4) and intratumoral conditions (pH 5.5, carboxyl-esterase) (FIG. 7B). Cabo-OH is linked to the dendrimer through an ester bond via its hydroxyl group at position, 7 allowing for pH and esterase-responsive release. This design facilitates controlled release in both intracellular and intratumor environments, effectively restricting drug exposure beyond the boundaries of the prostate tumor. In PBS buffer at pH 7.4, we observed a <40% drug release in about 2 weeks. Notably, only 10% of the drug was released within the first 8 hours. However, under intracellular conditions, the PD-CTT1298-Cabo conjugate demonstrated a gradual and sustained release of the drug over 2 weeks. Around 15% drug was released in first 8 hours, gradually increasing to ˜35% in 48 hours, reaching approximately 80% drug release over a 12-day period (FIG. 7B). This sustained intracellular release profile is favourable for delivering Cabo-OH through PD-CTT1298-Cabo conjugate to prostate cancer cells for prostate cancer treatment.

### PD-CTT1298-Cabo Conjugate Improves the Anti-Proliferative Activity of Free Cabo and Cabo-OH

To study the efficacy of the PD-CTT1298-Cabo conjugate in comparison to Cabo and Cabo-OH, a cell viability experiment was performed (FIG. 7D). PC3-PIP PSMA (+) cells were incubated with different concentrations of the compounds along with 10% DMSO which served as a control. Treatments were administered at equivalent drug bases. The cells were incubated for 72 H and the luminescence of the viable cells was measured using the Cell-Titer-Glo® Luminescent Cell Viability assay. As expected, the 10% DMSO led to almost no viable cells present. Both dendrimer-Cabo conjugate (PD-CTT1298-Cabo) and free drugs (Cabo & Cabo-OH) exhibited dose dependent efficacy. At the highest concentration tested (1000 μg/mL), the PD-CTT1298-Cabo was significantly more potent than free Cabo & Cabo-OH. The same results were observed at 100 μg/mL. However, at lower concentrations the dendrimer conjugate was comparable to the free drugs. There was no significant difference observed between the dendrimer and the free drugs with the highest percentage of viable cells present after 72 H. Overall, the dendrimer was more effective at inducing apoptosis in PSMA (+) cells compared to the free drugs. PD-CTT1298-Cabo exhibited a lower IC₅₀ of 9.83±1.11 μg/mL compared to 24.83±2.27 μg/mL of Cabo (p=0.00012 free Cabo vs. PD-CTT1298-Cabo), and 77.30±4.35 μg/mL of Cabo-OH (p=0.0013 Cabo-OH vs. PD-CTT1298-Cabo) indicating stronger anti-proliferative activity. Given the findings from biodistribution and this in vitro experiment, it is contemplated that the in vivo targeting of PD-CTT1298-Cabo to prostate tumor could lead to markedly enhanced therapeutic outcomes for treating prostate cancer.

### Conclusions

In this study, we have developed a PSMA-targeted dendrimer platform using a highly efficient SPAAC chemistry, for PSMA (+) prostate cancer specific targeted drug delivery. We show that the conjugation of an irreversible PSMA inhibitor, CTT-1298 to dendrimer retains its PSMA-binding ability in nanomolar range. The PSMA targeted dendrimer specifically localizes in PSMA (+) prostate cancer cells in vitro and in prostate tumor in vivo in a PC-3 PIP tumor xenograft mouse model. While the dendrimer remains in the tumor for 48 hours, it clears rapidly from the peripheral organs limiting systemic side-effects. We further developed PSMA-targeted dendrimer cabozantinib conjugate, PD-CTT1298-Cabo, that exhibited a nanomolar c-Met inhibitory activity. Further, dendrimer based PSMA targeting, and triggered and sustained intra-tumoral drug release significantly improved the anti-proliferative activity of PD-CTT1298-Cabo in PSMA (+) prostate cancer cells. These results indicate that utilizing PSMA-targeted dendrimer-based delivery may improve efficacy and widen the therapeutic window of cabozantinib and other chemotherapeutic agents. This approach may be particularly beneficial in addressing concerns associated with dose-related systemic toxicities of chemotherapies for the treatment of prostate cancer.

### EXAMPLE 2

Here we present a novel therapeutic approach using PSMA-targeted 2-deoxyglucose-dendrimer (PSMA-2DG-D) for targeted delivery of a potent tyrosine kinase inhibitor, cabozantinib (Cabo), selectively to PC cells. PSMA-2DG-D demonstrates intracellular localization in PSMA (+) PC cells through PSMA-mediated internalization. This PSMA-specific targeting translates to enhanced efficacy of Cabo compared to the free drug when conjugated to PSMA-2DG-D. Furthermore, systemically administered fluorescently labeled PSMA-2DG-D-Cy5 specifically targets PSMA (+) tumors with minimal off-target accumulation in PC3-PIP tumor xenograft mouse model. This demonstrates that the PSMA-2DG-D platform is a promising new delivery system for potent chemotherapeutics, where systemic side effects are a significant concern.

### Materials and Methods

### Synthesis of PSMA-2DG-D-Cy5 and PSMA-2DG-D-Cabo Conjugates

### Materials and Reagents

Reagents were purchased from commercial suppliers and used without further purification unless otherwise stated. Thin layer chromatography (TLC) was performed using aluminum sheets precoated with silica gel 60 F₂₅₄ (0.2 mm, Merck). The Chromatographic spots were visualized either using UV light or spots were stained using various reagents for better visualization. Merck silica gel 60 (230-400 mesh) was used for column chromatography. Dialysis was performed using Spectra/Por dialysis membranes purchased from Repligen. Triton® X-100 was procured from Aaron Chemicals. PBS was purchased from Cytiva. Rat Sprague Dawley red blood cells (RBCs) were purchased from Innovative Research.

### Instruments

¹H (500 MHz), ¹³C (125 MHz), and ³¹P (202 MHz) NMR were obtained on a Bruker 500 MHz spectrometer. Spectra were recorded at ambient temperature and the chemical shifts are reported as δ values (ppm) referenced to δ 7.26 ppm and δ 77.1 ppm for CDCl₃, δ 3.31 ppm and 49.00 for MeOH-d₄, δ 4.79 for D₂O. Coupling constant (J) is reported in Hertz (Hz). Patterns of Splitting are designated as s: singlet, d: doublet, t: triplet, dd: double doublet, m: multiplet, ddd: doublet of doublet of doublet, and br: broad peak. High-Resolution Mass Spectra (HRMS) were recorded on a Bruker-micrOTOF-Q II spectrometer using ESI as the ion source. The purity and drug release studies were analyzed using high-performance liquid chromatography (HPLC). The HPLC was performed using a Waters Acquity Arc® system (Milford, MA, USA), equipped with binary pumps, 2998 PDA detector, and a 2475 fluorescence detector. The analyses were performed using Waters Empower software. The samples were run using Waters C18 symmetry 300 (5 μm) 4.6×250 mm column using a gradient flow method. Two different HPLC methods were used as follows. HPLC Method A: The method started with 90:10 (Solvent A: 0.1% TFA in water; Solvent B: 0.1% TFA in ACN), gradually increased to 50:50 (A:B) at 20 minutes, 10:90 (A:B) at 38 minutes, and finally returned to 90:10 (A:B) at 40 minutes. HPLC Method B: The method started with 80:20 (Solvent A: 0.1% TFA in water; Solvent B: 0.1% TFA in ACN), gradually increased to 30:70 (A:B) at 15 minutes, 50:50 (A:B) at 25 minutes, and finally returned to 80:20 (A:B) at 30 minutes. A flow rate of 1 mL/min was maintained during the run. In both methods, a flow rate of 1 mL/min was maintained during the run. The dendrimers and drug were detected at 205, 210, 305, or 331 nm. The Cy5 labeled conjugate was detected at 650 nm. The size and zeta potential distribution of dendrimers were determined using a Malvern Zetasizer® Pro Blue instrument (Malvern Panalytical). For size distribution analysis, dendrimer samples were dissolved in milli Q water at a concentration of 0.1 mg/ml, while for zeta potential distribution analysis, samples were dissolved in a 10 mM sodium chloride solution at a concentration of 0.2 mg/ml. Measurements were conducted in triplicate, and the obtained data were averaged to yield the reported sizes and zeta potential values.

### Synthesis Procedures

Synthesis of 2DG-D: The synthesis and characterization of 2DG-D was carried out using our recently published procedure. The structure of 2DG-D is presented in FIG. 8.

Synthesis of compound 2a: 6-azido-hexanoic acid (5.7 mg, 8.0 eq, 0.036 mmol) in Anhy. DMF (5 ml) was activated by adding EDC.HCl (13 mg, 25.0 eq, 0.067 mmol) and stirred for 10 minutes. To stirred solution of 2DG-D 1 (100 mg, 1.0 eq, 0.0045 mmol) in DMF (5 mL) was added azido-hexanoic acid solution dropwise followed by the addition of DMAP (2.7 mg, 5.0 eq, 0.022 mmol) and the reaction mixture was stirred for 24 hours at room temperature. The reaction completion was confirmed by the shift in the retention time of the chromatogram in HPLC. The dialysis was performed using a 1 kDa dialysis membrane in DMF for 12 h. The final dialysis was performed against DI water. The product was lyophilized to afford compound 2a in 85% yield.

¹H NMR (500 MHz, DMSO) δ 8.54 (s, 8H, triazole-H), 8.24-7.85 (m, 52H, triazole-H+amides-H), 7.24 (s, 16H, Ar-H), 4.99-4.80 (m, sugar-H), 4.64-4.45 (m, sugar-H), 4.23-4.07 (m, sugar-H), 3.94-3.78 (m, sugar-H+PEG-H), 3.76-3.51 (m, PEG-H), 3.19-3.06 (m, PEG-H), 2.77-2.62 (m, core-CH₂), 2.39-2.33 (m, 14H, azido-linker-CH₂), 2.30-2.13 (m, core-CH₂), 1.98-1.79 (m, 40H, sugar-H+core-CH₂), 1.68-1.44 (m, linker-CH₂), 1.42-1.34 (m, 12H, azido-linker-CH₂).

Synthesis of compound 2b: 6-azido-hexanoic acid (21.5 mg, 15.0 eq, 0.137 mmol) in Anhy. DMF (5 ml) was activated by adding EDC.HCl (43 mg, 25.0 eq, 0.227 mmol) and stirred for 10 minutes. To stirred solution of 2DG-D 1 (200 mg, 1.0 eq, 0.009 mmol) in DMF (5 mL) was added azido-hexanoic acid solution dropwise followed by the addition of DMAP (7.8 mg, 7.0 eq, 0.06 mmol) and the reaction mixture was stirred for 24 hours at room temperature. The reaction completion was confirmed by the shift in the retention time of the chromatogram in HPLC. The dialysis was performed using a 1 kDa dialysis membrane in DMF for 12 h. The final dialysis was performed against DI water. The product was lyophilized to afford compound 2b in 82% yield.

¹H NMR (500 MHZ, DMSO) δ 8.47 (s, 8H, triazole-H), 8.12-7.73 (m, 52H, triazole-H+amides-H), 7.17 (s, 16H, Ar-H), 5.22-5.03 (m, Sugar-H), 4.92-4.71 (m, Sugar-H), 4.58-4.33 (m, Sugar-H), 4.31-4.02 (m, Sugar-H+PEG-H), 3.86-3.72 (m, PEG-H), 3.66-3.44 (m, PEG-H), 3.34-3.15 (m, PEG-H), 3.11-2.99 (m, core-CH₂), 2.68-2.53 (m, core-CH₂), 2.34-2.25 (m, 24H, azido-linker-CH₂), 2.24-2.16 (m, core-CH₂), 2.16-2.06 (m, core-CH₂), 1.93-1.72 (m, 40H, Sugar-H+core-CH2), 1.62-1.48 (m, linker-CH₂), 1.49-1.38 (m, 24H, Sugar-H), 1.37-1.27 (m, 24H, azido-linker-CH₂).

Synthesis of compound 3: To solution of 2DG-D-azide (2) (20.0 mg, 1.0 eq, 0.0012 mmol) in DI Water (150 μL) was added DBCO-C6-CTT1298 (5.5 mg, 4.0 eq, 0.005 mmol) and the reaction mixture was stirred for 12 hours at room temperature. Reaction progress was tracked with HPLC. Upon completion, the dialysis was performed using a 1 kDa dialysis membrane in DI Water for 12 h. The aqueous solution was lyophilized to afford PSMA-2DG-D (3) in 90% yield.

¹H NMR (500 MHz, D₂O) δ 8.11-7.88 (m, 23H, triazole-H+ligand-Ar-H), 7.80-7.12 (m, 34H, ligand-Ar-H), 7.07 (s, 16H, Ar-H), 4.99-4.91 (m, ligand-H), 4.61-4.37 (m, sugar-H+ligand-H), 4.21-4.00 (m, sugar-H+ligand-H), 3.93-3.40 (m, sugar-H+PEG-H), 3.33-3.15 (m, PEG-H), 3.12-2.94 (m, core-CH₂), 2.87-2.48 (m, core-CH₂), 2.36-1.93 (m, azido-linker-CH₂+ligand-H), 1.90-1.72 (m, 40H, sugar-H+core-CH₂), 1.68-1.59 (m, sugar-H), 1.57-1.30 (m, azido-linker-CH₂+ligand-H), 1.32-1.08 (m, ligand-H). ³¹P NMR (202 MHz, D₂O) δ 7.10.

Synthesis of compound 4: PSMA-2DG-D (3) (19.0 mg, 1.0 eq, 0.0001 mmol) was dissolved in DI Water (150 μL) and stirred. Cy5-DBCO (2.77 mg, 3.0 eq, 0.003 mmol) was added to the dendrimer solution and the stirring mixture was left for 48 h at room temperature. The completion of reaction was tracked using HPLC. Upon completion, the dialysis was performed using a 1 kDa dialysis membrane in DI Water for 12 h. The aqueous solution was lyophilized to afford compound PSMA-2DG-D-Cy5 (4) in 81% yield.

¹H NMR (500 MHz, DMSO) δ 8.46-8.13 (m, 15H, Cy5-H), 8.05-7.87 (m, 38H, ligand-H+triazole-H), 7.85-7.70 (m, 18H, ligand-H+Cy5-H), 7.69-7.14 (m, ligand-H+core-H+Cy5-H), 7.11 (s, 16H, Ar-H), 6.54 (t, J=12.5 Hz, 1H, Cy5-H), 6.26 (dd, J=26.6, 13.7 Hz, 2H, Cy5-H), 5.85 (ddd, J=55.9, 17.3, 8.2 Hz, 3H, Cy5-H), 5.27-4.64 (m, ligand-H+sugar-H), 4.65-4.11 (m, sugar-H+ligand-H), 4.11-3.94 (m, sugar-H+ligand-H), 3.83-3.63 (m, sugar-H+PEG-H), 3.62-3.44 (m, PEG-H), 3.43-3.09 (m, PEG-H), 3.06-2.65 (m, ligand-H+core-H+Cy5-H), 2.64-2.47 (m, core-H+ligand-H+Cy5-H), 2.42-2.30 (m, core-H), 2.28-2.19 (m, ligand-H+azido-linker-CH₂), 2.17-1.99 (m, Cy5-H+core-H+azido-linker-CH₂), 1.98-1.93 (m, Cy5-H), 1.92-1.63 (m, sugar-H+core-CH₂), 1.54-1.32 (m, azido-linker-CH₂+ligand-H), 1.30-0.68 (m, ligand-H+Cy5-H).

Synthesis of compound 7: To a solution of 2DG-D-azide (2) (20 mg, 1.0 eq, 0.0013 mmol) in DMF (100 μL) in 2 mL vial, was added solution of compound DBCO-Cabo (6) (7.8 mg, 8.0 eq, 0.010 mmol) dissolved in DMF (100 μL). The reaction mixture was stirred at RT for 3 h. The reaction completion was confirmed by the shift in the retention time of the chromatogram in HPLC. Upon completion, the compound was purified with 3 kDa dialysis membrane. The product was lyophilized to afford 2DG-D-Cabo (7) in 88% yield. White fluffy Solid.

¹H NMR (500 MHz, DMSO-d₆) δ 10.34-10.21 (s, Cabo-amide-H), 10.17-10.05 (s, Cabo-amide-H), 8.66-8.46 (m, triazole-H+Cabo-Ar-H), 8.17-8.06 (2DG-D-Ar-H+Cabo-Ar-H), 8.04-7.96 (Cabo-Ar-H), 7.96-7.80 (2DG-D-triazole-H+Cabo-Ar-H), 7.80-7.52 (2DG-D-Ar-H+core amide-H+Cabo-Ar-H), 7.52-7.41 (2DG-D-Ar-H+Cabo-Ar-H), 7.41-7.26 (s, Cabo-Ar-H), 7.26-7.22 (2DG-D-Ar-H+Cabo-Ar-H), 7.22-7.17 (s, Cabo-Ar-H), 6.64-6.52 (m, DBCO-CH₂), 6.10-5.92 (m, Cabo-H), 5.28-5.14 (m, DBCO-CH₂), 5.08-4.75 (m, sugar-H+Cabo-H), 4.74-4.40 (m, sugar-H+Cabo-H), 4.39-4.26 (m, sugar-H+Cabo-H), 4.27-4.03 (m, sugar-H+Cabo-H), 4.03-3.89 (m, PEG-H+sugar-H+Cabo-H), 3.89-3.75 (m, PEG-H+Cabo-H), 3.74-3.45 (m, PEG-H+Cabo H), 3.26-3.03 (m, sugar-H+Cabo-H), 2.81-2.57 (m, sugar-H+Cabo-H), 2.43-2.07 (m, azido-linker-H+Cabo-H), 2.01-1.71 (m, azido-linker-H+Cabo-H), 1.70-1.21 (m, azido-linker-H+Cabo-H).

¹³C NMR (125 MHz, DMSO-d₆) δ 172.5, 170.8, 168.6, 166.0, 160.7, 152.2, 150.8, 144.3, 142.6, 140.4, 137.1, 135.7, 130.6, 129.7, 128.1, 125.5, 123.9, 123.5, 122.4, 122.2, 122.0, 121.1, 119.8, 116.3, 116.1, 114.9, 114.8, 107.3, 106.0, 101.6, 97.8, 96.5, 74.1, 73.0, 72.7, 72.3, 71.5, 71.3, 70.5, 70.2, 70.0, 69.4, 69.1, 68.3, 68.0, 67.7, 67.2, 66.0, 65.1, 64.0, 62.8, 62.6, 61.5, 60.4, 57.2, 56.0, 50.9, 49.7, 48.6, 40.9, 40.8, 38.3, 37.3, 35.3, 32.0, 30.4, 29.4, 25.1, 15.9, 14.6.

Synthesis of compound 8: To a solution of 2DG-D-Cabo 6 (20 mg, 1.0 eq, 0.0009 mmol) in deionised water (100 μL) in a 2 ml glass vial was added solution of DBCO-PSMA (2.8 mg, 3.0 eq, 0.0028 mmol) dissolved in DI water (50 μl). The completion of reaction was tracked using HPLC. Upon completion, the product was lyophilized to afford PSMA-2DG-D-Cabo (7) in 91% yield. White fluffy Solid.

¹H NMR (500 MHz, DMSO-d₆) δ 10.39-9.88 (m, Cabo-amide H), 8.6-8.4 (m, triazole-H+Cabo-Ar-H), 8.1-7.9 (m, 2DG-D-Ar-H+Cabo-Ar-H), 7.9-7.7 (m, 2DG-D-Ar H+ligand-Ar-H+Cabo-Ar-H, core amide H), 7.7-7.5 (m, 2DG-D-Ar-H+ligand-Ar H+Cabo-Ar-H), 7.5-6.9 (m, 2DG-D-Ar-H+ligand-Ar-H+Cabo-Ar-H), 6.6-6.4 (s, Cabo-Ar-H), 6.1-5.8 (m, Cabo-Ar-H), 5.2-4.7 (m, 2DG-D-OH+Cabo-Ar-H), 4.7-4.2 (m, sugar-H+ligand H+Cabo-H), 4.2-3.7 (m, sugar-H+ligand-H+Cabo-H), 3.7-3.4 (m, Cabo-H+PEG-H), 3.2-2.7 (m, sugar-H+PEG-H), 2.7-2.5 (m, ligand H+Cabo-H+sugar-H), 2.3-1.6 (m, azido-linker-H +Cabo H), 1.6-0.8 (m, azido-linker-H+Cabo H+Cabo-H+ligand-H).

³¹P NMR (202 MHz, D₂O) δ 7.35.

### PSMA IC₅₀

As previously reported by our laboratory, the standard procedure for determining the IC₅₀ for PSMA-2DG-D-Cy5 involved utilizing concentrations of 80, 40, 20, 10 and, 5 nM. The results are displayed as the average value along with the standard error of the mean (SE).

### In Vitro Drug Release Studies

In vitro drug release investigations were conducted under simulated plasma conditions (phosphate-buffered saline, PBS, pH 7.4) and intra-tumoral conditions (citrate buffer, pH 5.5, supplemented with esterase). The PSMA-2DG-D-Cabo conjugate was dissolved at a concentration of 1 mg/ml in each respective buffer and subjected to incubation at 37°° C. with continuous agitation to mimic physiological conditions. At specified intervals, samples were withdrawn, immediately quenched with an equivalent volume of methanol, and subsequently stored at −20° C. until further analysis. The liberated drug was subsequently analyzed using high-performance liquid chromatography (HPLC), with the extent of drug release determined by comparing it to the standard curve established for the free drug (Cabo-OH) on the HPLC system.

### In Vitro Uptake and Viability Studies

Cell culture. The PC3-PIP (+) PSMA and PC3 (−) PSMA cell lines were grown in complete culture media composed of RPMI-1640 (VWR, 16750) with 2 mM L-glutamine, 100 U/ml PenStrep (Cytiva, SV30010), and supplemented with 10% HI-FBS (VWR, 97068). Media was changed every 3-4 days. Cells were passaged upon reaching 80% confluence. Trypsin protease solution (Cytiva, SH30236) was used per manufacturer instructions to release cells before passaging which involved changing media, counting, and seeding 1×10⁵ cells in 20 ml of new complete media in a new T-75 culture flask (VWR, 10062).

Flow cytometry. To determine whether the PSMA-2DG-D-Cy5 dendrimer was selectively targeted to PSMA (+) cells, a cell uptake experiment was performed. 5×10⁵ PC3-PIP (+) PSMA cells and PC3 PSMA (−) cells were added to individual Eppendorf vials. Cells were then treated with 1 ug/ml (50 nM) of the fluorescently labeled PSMA targeted dendrimer at various time points (T=0, 0.5, and 1 h). Following incubation at 37° C., cells were centrifuged at 1200 RPM for 2 min and the supernatant was discarded. The samples were washed 3× with ice cold PBS, fixed with 4% formaldehyde at RT for 15 min. Following fixing the cells, the samples were centrifuged at 1200 RPM for 2 min, washed 3× with ice cold PBS and suspended in FACS Buffer for analysis via flow cytometry. An Attune NxT® Flow Cytometer was used to analyze all the samples. Blank cells were run to set an appropriate gate and 10,000 events were collected per sample. The Mean Fluorescence Intensity (MFI) was acquired and normalized. For the blocking experiments, the same procedure was repeated except the cells were incubated with 500 nM of CTT 1057 (PSMA irreversible inhibitor) for 30 min prior to the addition of the dendrimer. For dose response studies, the dendrimer, at various concentrations was incubated for 1 h and analyzed. Statistical analyses were performed using a TWO-WAY ANOVA T-Test with unequal variances. Histograms were also analyzed for the shift in fluorescence intensity for all the experiments performed.

Confocal microscopy. 5×10⁵ PC3-PIP (+) PSMA cells and PC3 (−) PSMA cells were added to Nunc Lab-Tek II® Chamber Slide system (Thermo Scientific, 154526) glass microscopy slides and incubated overnight in complete media. Following incubation, the media was aspirated, and cells were treated with 50 μg/ml (2.5 μM) of the fluorescently labeled PSMA-2DG-D-Cy5 dendrimer for 30 min. Following incubation at 37° C., the media was discarded, and the slides were washed 3× with ice cold PBS (VWR, 16750). The cells were first fixed using sterile 4% Formaldehyde (Sigma Aldrich, 252549) at RT for 15 min. Following fixation, the cells were washed 3× with ice cold PBS and treated with 1 μg/ml (3.6 μM) DAPI at RT for 30 min. Following treatment of cells with DAPI, the supernatant was aspirated, and the cells were washed 3× with ice cold PBS. Finally, the slides were mounted using Vectashield Plus® Antifade Mounting Medium (Vector Laboratories, H-1900) and allowed to rest for 1 h at RT. The mounted slides were then analyzed and imaged using a Leica SP-8® Confocal Laser Scanning Microscope equipped with a water immersed 63× lens and multiple excitation lasers.

For deciphering the mechanism of these developed dendrimers, a similar cell uptake study was carried out with PC3-PIP (+) PSMA cells in presence of cell trafficking endocytosis inhibitors viz., chlorpromazine (CPZ), and methyl β-cyclodextrin (MβCD) for clathrin and caveolin mediated endocytosis inhibition, and GLUT inhibitor viz., cytochalasin B (10 μg/mL, 20 μM), following our previous report with few modifications.³³ In brief, 2×10⁵ cells were plated in a 6-well plate on the cover slips, and grown for overnight at 37° C. in a CO₂ incubator. The cells were washed using 1× PBS and then incubated in serum free medium containing chlorpromazine with CPZ (10 μg/mL, 30 μM), MβCD (2.5 mg/mL, ˜2 mM), for 1 h at 37° C. in a CO₂ incubator. The media was aspirated, and the cells were washed with PBS and incubated with the PSMA-2DG-D-Cy5 and 2DG-D-Cy5 for 4 h at 37° C. Following this, the cells were again washed with ice cold PBS several times and then fixed with 4% paraformaldehyde (PFA) for 15 min at 37° C. The PFA was removed, and the cells were washed with cold PBS again and then these cells were permeabilized by treating with 0.1% of triton-X-100 for ˜15 min followed by its washing. The cells were finally stained with 10 μL of (50 μM) DAPI for 20 min and imaged using Leica SP-8 Confocal Laser Scanning Microscope at 20× magnification.

Cell viability. 2.5×10⁴ PC3-PIP PSMA (+) cells were allowed to adhere to a 96 well plate overnight. The following day, cell media was changed and the PSMA-2DG-D-Cabo dendrimer, at the concentrations tested, were added along with Cabozantinib (Cabo) and Cabozantinib-7-hydoxy derivative (Cabo-OH). The cells were allowed to incubate at 37° C. for 72 h. Following incubation, the luminescence of the viable cells was measured using the CellTiter-Glo® Luminescent Cell Viability assay (Promega, G7570) according to manufacturer's instructions. The luminescence data was collected using a plate reader. Cell viability (%) was calculated using the luminescence values obtained from the controls used in the experiment. The blank cells served as a positive (+) control while sterile 10% DMSO (Fisher Scientific, BP231) served as the negative (−) control. The experiment was performed in triplicate. A similar experiment was carried out with 2DG-D dendrimer at different concentrations (0.1, 1, 10, 100 and 1000 μg/ml) following the same protocol. The experiments were performed in triplicate, with proper controls. Statistical analyses were performed using a TWO-WAY ANOVA T-Test with unequal variances.

### Hemocompatibility Assay

To evaluate the compatibility of the dendrimer with the blood before in vivo studies, haemolysis assay was done on the red blood cells (RBCs) from Sprague Dawley rats following the previous published reports with minor modifications. For this, 5 ml of 1:1 solution of RBC:1x PBS was made and from this, ˜250 μl was taken and put in micro centrifuge tubes along with same volume of PSMA-2DG-D-Cy5 dendrimer at different concentrations (5, 2.5, 1.25, 0.63 and 0.31 mg/ml). The samples were further incubated at 37° C. for 3 h with shaking using an incubator shaker. All the samples were then centrifuged for 10 min (5000 rpm), and ˜250 μl of supernatant from each sample was put in a 96 well plate and absorbance measurements were taken at 540 nm, using the Thermo Scientific Multiskan SkyHigh® Microplate reader. The percentage haemolysis was calculated using equation 1, and according to the ASTM E2524-08 standards, a material showing less than 5% hemolysis is considered as hemocompatible.

$$Hemolysis ( \% ) = \frac{Absorbance of the test sample - Absorbance of the negative control}{Absorbance of the positive control - Absorbance of the negative control} \times 100 \qquad ( 1 )$$

### In Vivo Fluorescence Studies

All animal studies received prior approval with AFAF #6635 from the IACUC at Washington State University, with all methods for animal studies following ARRIVE guidelines. The PC3 PSMA−/+ cell line was cultured in RPMI containing 10% fetal bovine serum and 1% penicillin-streptomycin. 5-week-old male thymus-less nude mice were purchased from The Jackson Laboratory. 2×10⁶ PC3 PSMA−/+ cells were mixed with Matrigel® (BD Biosciences) at a 1:1 ratio and then injected subcutaneously into nude mice (3 nude mice per group). Once the tumor volume reached 200 mm³, all PC3-bearing nude mice were injected intravenously with PSMA-2DG-D-Cy5 at a dose of 20 mg/kg (100 μL). The whole-body fluorescence distribution was observed using an in vivo imaging system (IVIS) at 1 h, 6 h, 24 h and 48 h after injection. Mice were then sacrificed and dissected to obtain ex vivo fluorescence images of major organs. The fluorescence intensity of the region of interest (ROI) was calculated by the Indigo software accompanying IVIS.

Statistical analyses: Statistical analyses were performed using a TWO-WAY ANOVA T-Test with unequal variances via GraphPad Prism®.

### Results and Discussion

### Synthesis of Fluorescently Labelled PSMA-2DG-D-Cy5 Conjugates Using SPAAC Chemistry

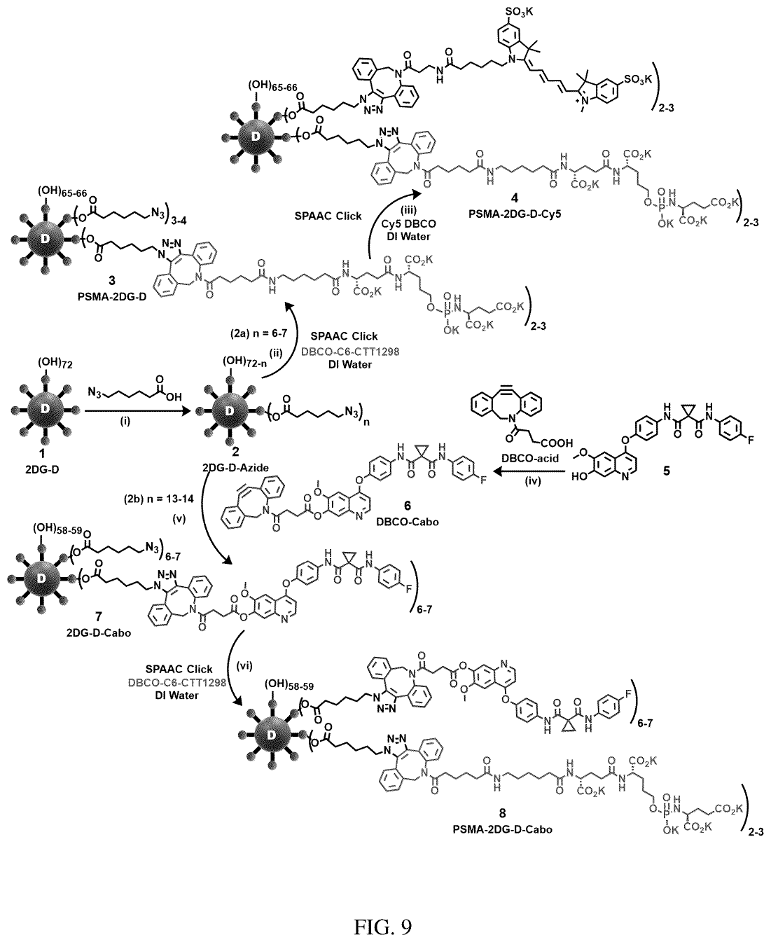

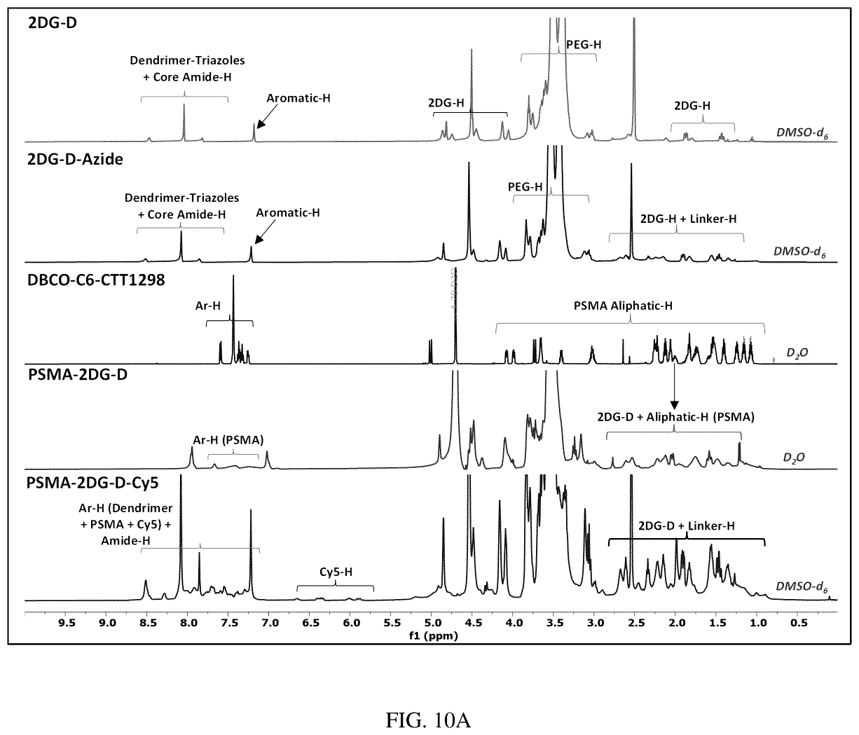

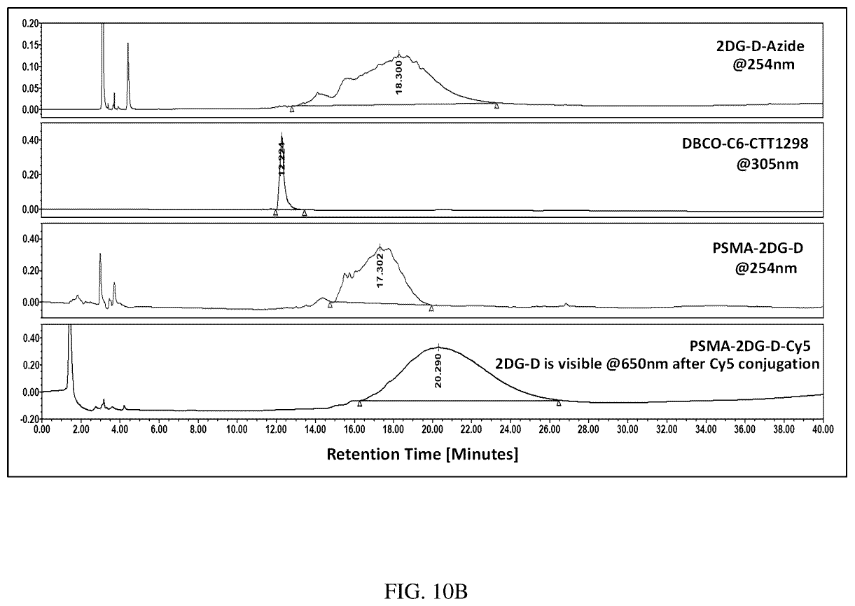

We first developed the fluorescently (Cyanine 5) labelled PMSA-targeted dendrimer platform (PSMA-2DG-D-Cy5) to evaluate the in vitro and in vivo uptake and mechanism of uptake in PSMA (+) PC cells via confocal microscopy and in vivo fluorescence imaging. The synthesis and structural characterization of 2DG-D was performed using our recently published procedure and the structure of 2DG-D is presented in FIG. 8. The synthesis pathway of PSMA-2DG-D-Cy5 initiated with the partial esterification of the peripheral hydroxyl groups of 2DG-D (1) through the reaction with azido-hexanoic acid employing Steglich esterification to obtain 2DG-D-azide (2a). The synthesis scheme is depicted in FIG. 9. The successful incorporation of six azido-linkers was confirmed by comparing the linker protons between δ 1-2 ppm with aromatic protons of 2DG-D dendrimer at δ 7.24 ppm using proton integration method (FIG. 10A). Subsequently, the coupling of 2DG-D-azide (2a) dendrimer and PSMA ligand, DBCO-C6-CTT1298 was achieved via the SPAAC reaction in deionized (DI) water to afford PSMA-2DG-D (3). The number of PSMA ligand attachment was confirmed through the comparative integration of aromatic protons from the ligand, between δ 7-8 ppm, and the 16 aromatic protons of the dendrimer at δ 7.06 ppm (FIG. 10A). Additionally, the ³¹P NMR spectrum further substantiated the successful conjugation of the PSMA ligand on to the dendrimer (data not shown). Analysis of the HPLC chromatogram showed the expected shift in retention time, from 18.3 minutes for 2DG-D-azide to 17.3 minutes for PSMA-2DG-D upon conjugation with the PSMA targeting ligand (FIG. 10B). Furthermore, the HPLC purity of PSMA-2DG-D surpassed 97% (FIG. 10B). Subsequent SPAAC reaction of PSMA-2DG-D with Cy5-DBCO yielded the final fluorescent dendrimer PSMA-2DG-D-Cy5 (4). The confirmation of Cy5 attachment was achieved through the presence of Cy5 protons in the ¹H NMR spectrum, with approximately two Cy5 molecules attached on the dendrimer surface (FIG. 10A). HPLC analysis showed purity exceeding 99%, with a significant shift in retention time from 17.3 to 20.0 minutes upon Cy5 conjugation. The characterization of all intermediates and final conjugates was conducted using NMR (¹H and ¹³C), and purity was assessed via HPLC (data not shown). To verify whether the dendrimer-bound PSMA retained its efficacy as a PSMA inhibitor, we performed the PSMA-IC₅₀ assessment of PSMA-2DG-D-Cy5 according to our previously reported protocol. The IC₅₀ value of PSMA-2DG-D remained within the nanomolar range (15.3 nM) (data not shown). This indicates that the attachment of PSMA ligand to the dendrimer did not compromise its ability to target and inhibit PSMA.

### Synthesis of Drug Conjugated PSMA-2DG-D-Cabo Conjugates Using SPAAC Chemistry

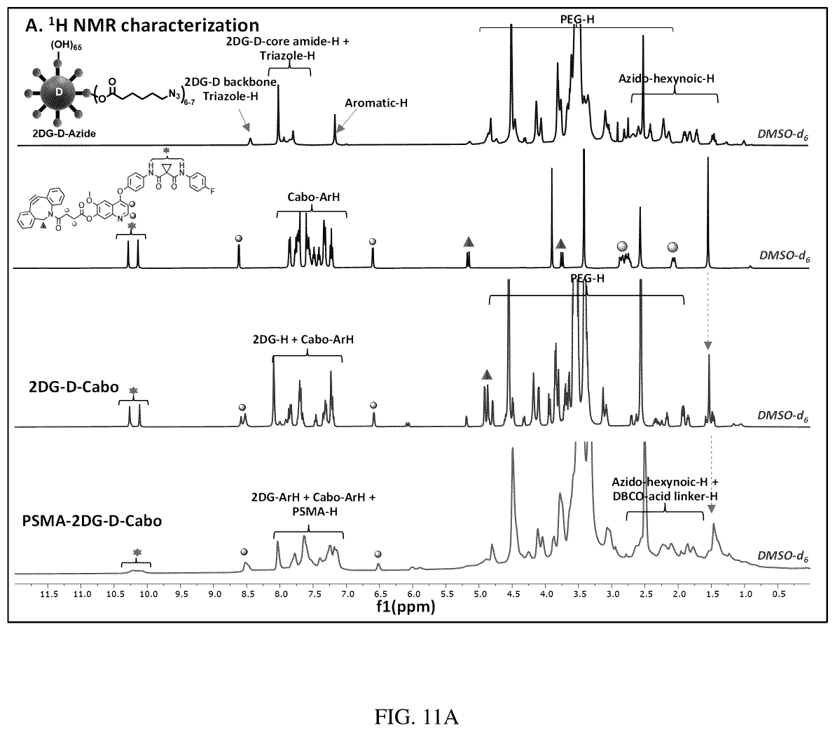

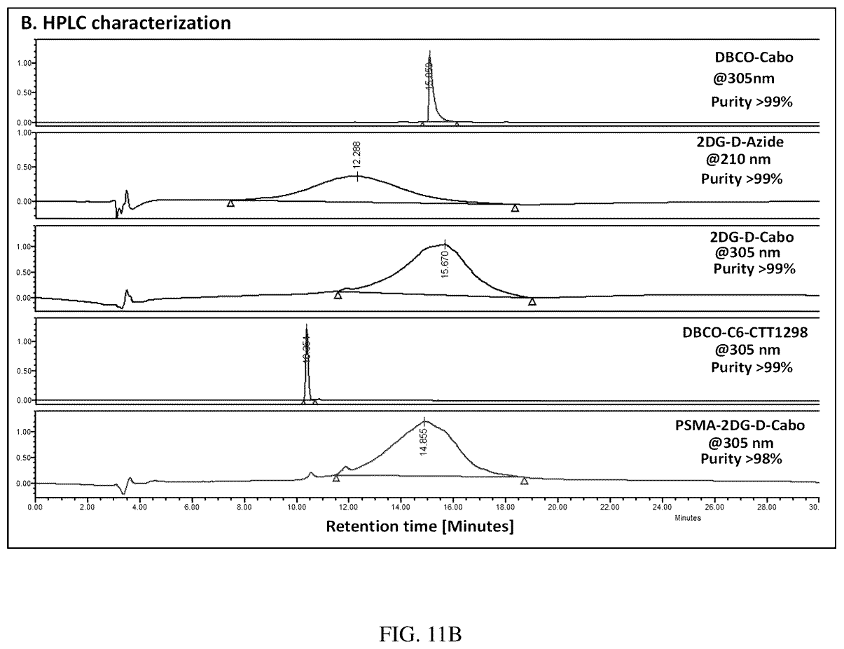

Further, to explore the potential of utilizing PSMA-2DG-D as a carrier for the delivery of Cabo to PSMA (+) PC cells, we developed PSMA-2DG-D-Cabo conjugate (FIG. 9). The Cabo was attached using enzyme and acid cleavable ester linkages capable of releasing the drug under intracellular and intra-tumoral environment. The synthesis of the PSMA-2DG-D-Cabo began with the preparation of DBCO-modified Cabo (DBCO-Cabo) using our previously reported protocol. Concurrently, 2DG-D underwent modification to introduce approximately 12 azide groups, yielding 2DG-D-azide (2b). The number of azide residues were estimated using proton integration method by comparing the integration of linker protons in between δ 1-2 ppm with aromatic protons of 2DG-D dendrimer at δ 7.17 ppm (data not shown). The 2DG-D-azide (2b) was further subjected to a SPAAC reaction in DMF with DBCO-Cabo (6), leading to the formation of the 2DG-D-Cabo conjugate (7). The verification of the successful Cabo conjugation onto the dendrimer scaffold was done using ¹H NMR spectroscopy, wherein the characteristic peaks associated with Cabo were present alongside the dendrimer protons (FIG. 11A). Specifically, distinct amide proton peaks of Cabo at δ 10.2 and 10.3 ppm, in conjunction with the doublet of quinoline ring protons of Cabo at 6.5 ppm were observed. Furthermore, the ¹H NMR analysis confirmed the attachment of approximately 7 drug molecules per dendrimer, corresponding to ˜10 weight percent drug loading (FIG. 11A). The SPAAC reaction enabled the conjugation of Cabo with an exact stoichiometric equivalent as analysed by the NMR, without requiring DBCO-Cabo in excess. A few chemical transformations in literature are recognized for consistently delivering precise and reproducible drug loading, especially in multivalent systems. SPAAC appears to offer a solution to one of the primary challenges in achieving reproducible drug loading within the context of targeted drug delivery. Monitoring of the reaction progress was achieved via HPLC, which revealed a distinct chromatographic shift (from 12.28 to 15.67 min), indicative of the successful attachment of Cabo to the dendrimer surface (FIG. 11B). Following purification through dialysis and subsequent freeze-drying, the 2DG-D-Cabo conjugate was obtained in 92% yield. The purity of the 2DG-D-Cabo conjugate was determined to be approximately 99% by HPLC analysis (FIG. 11B). Following the successful attachment of Cabo to the dendrimer surface, the compound 7 was reacted with PSMA ligand, DBCO-C6-1298, resulting in the synthesis of the PSMA-2DG-D-Cabo (8) conjugate. The emergence of PSMA protons in the ¹H NMR spectrum provided evidence of the successful attachment of the PSMA ligand to the dendrimer (FIG. 11A). Additionally, a signal at δ 7.34 ppm in the ³¹P NMR spectrum further confirmed the presence of the PSMA ligand on the dendrimer surface (data not shown). The HPLC chromatogram displayed a shift from 15.63 to 15.02 minutes (FIG. 11B) after PSMA attachment and revealed a purity exceeding 98% for 2DG-PSMA-Cabo (FIG. 11B). The MALDI-ToF analysis further confirmed the molecular weight of the dendrimer as ˜31 kDa (data not shown). All intermediates and final dendrimer-drug conjugates were characterized using NMR spectroscopy and HPLC techniques (data not shown).

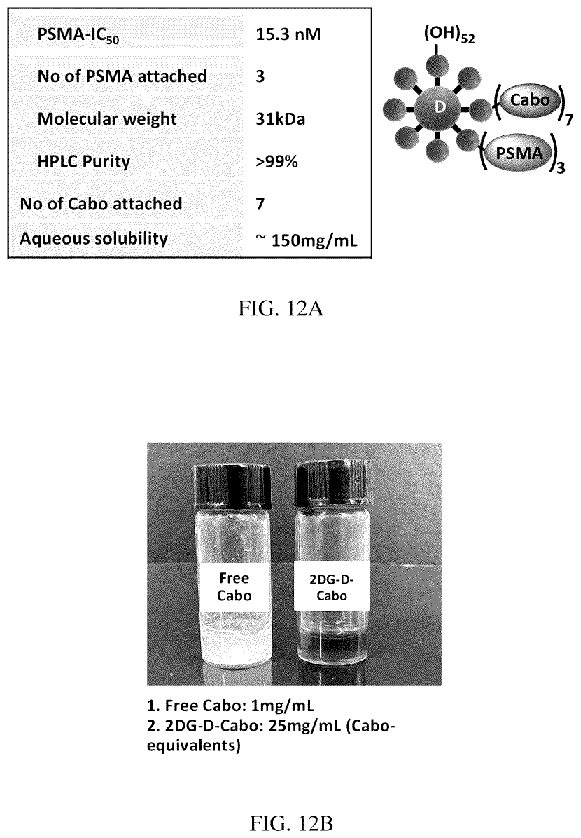

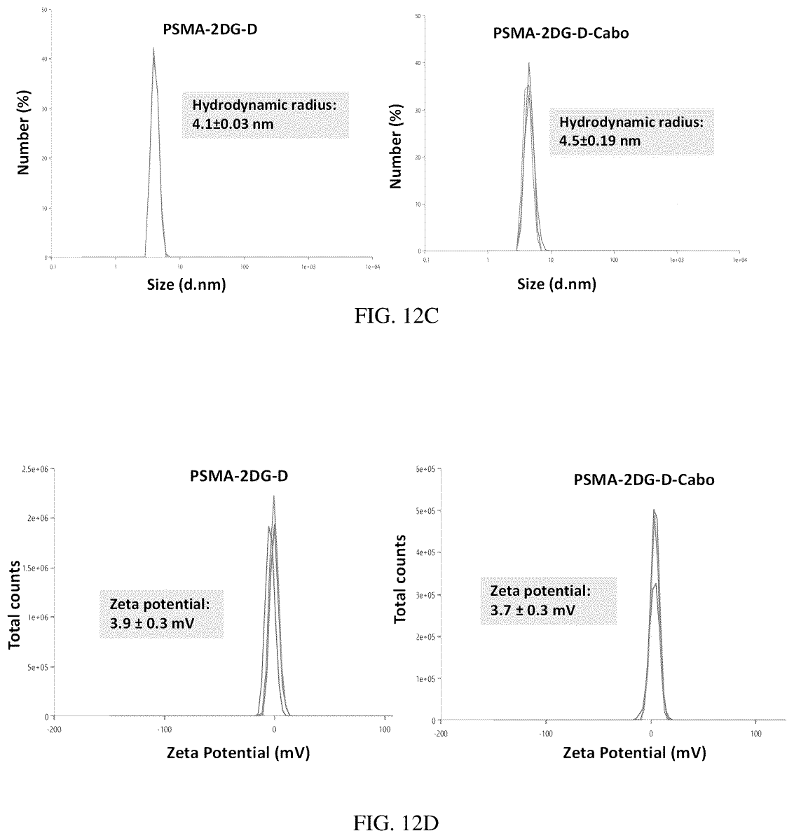

The physicochemical properties PSMA-2DG-D-Cabo are presented in FIG. 12A. Although both Cabo and Cabo-OH exhibit negligible solubility in water, the dendrimer conjugation of Cabo significantly boosted its solubility several folds (FIG. 12B). The aqueous solubility of PSMA-2DG-D-Cabo was determined to be approximately 150 mg/ml, translating to ˜15 mg/ml for Cabo. The hydrodynamic radius of PSMA-2DG and PSMA-2DG-D-Cabo measured 4.1±0.03 nm and 4.5±0.19 nm respectively (FIG. 12C-D), with a nearly neutral zeta potential distribution of 3.9±0.3 mV and 3.7±0.3 mV respectively as analysed by the dynamic light scattering (DLS) (FIG. 12C-D). Importantly, there was no significant alteration in the size of the PSMA-2DG-D dendrimer following Cabo conjugation, which is crucial for maintaining its clearance through renal filtration as demonstrated for 2DG-D and other similar sized dendrimers. Next, we assessed the comparative c-Met inhibition activities (IC₅₀) of PSMA-2DG-D-Cabo, that demonstrated nanomolar c-Met inhibitory activity (IC₅₀=1.67 nM).

### Fluorescently Labeled PSMA-2DG-D Demonstrates PSMA-Mediated Selective Uptake and Intracellular Localization in PSMA (+) PC Cells

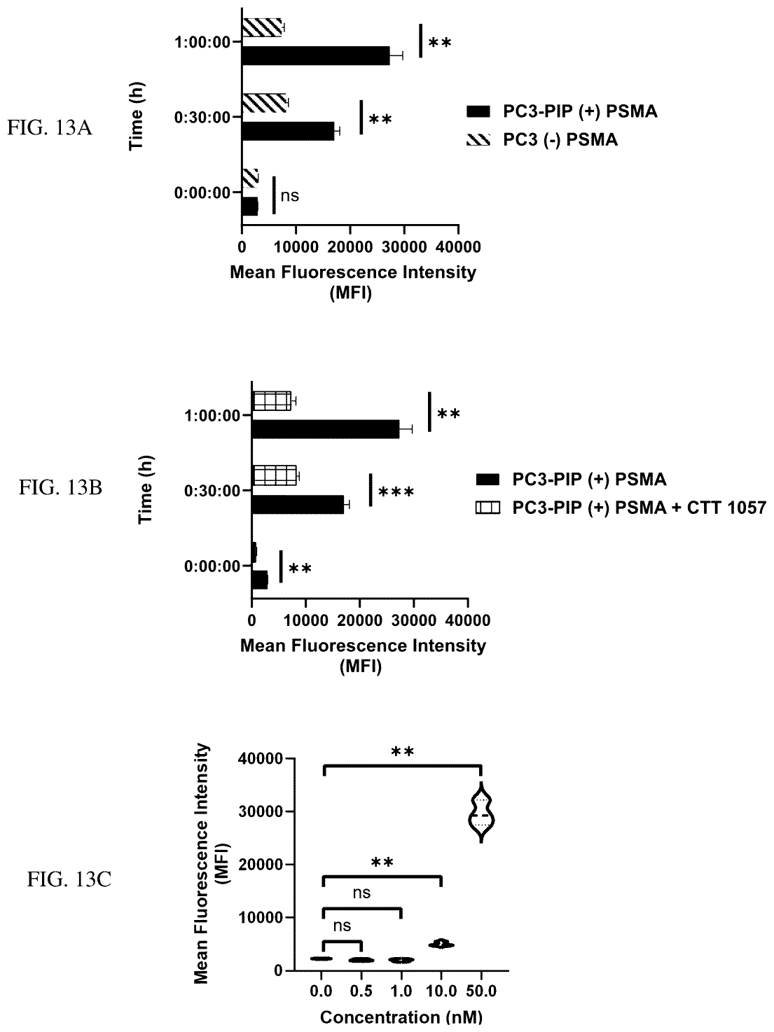

To study the targeting ability, selective uptake, and localization of the PSMA-2DG-D in PSMA (+) PC cells, the cell uptake experiments were performed in PC3-PIP (PSMA +) and PC3 (PSMA −) cells using flow cytometry and confocal microscopy (FIG. 13). At the 1 ug/mL (50 nM) concentration, selective uptake was observed in PSMA (+) PC3-PIP cells over 1 h (FIG. 13A). As expected, in PC3-PIP cells, the Mean Fluorescence Intensity (MFI) increased significantly over the time points which directly correlated to their high PSMA expression compared to PC3 cells, with the highest MFI observed at the 1 h time point (27347±2360). A significant change in MFI was observed in as little as 30 min (17077±997) when compared to the 0 h (2940±22; p<0.01) samples and increased over time until the final 1 h time point (p<0.01). Minimal uptake of the PSMA-2DG-D dendrimer was observed in PSMA (−) PC3 cells over 1 h, with no significant difference observed between the 0.5 h (8344±454) and 1 h time point (7478±542). The histograms obtained also showed a 2-log shift in the

PC3-PIP cells compared to PC3 cells (data not shown), which was anticipated based on their PSMA expression. Overall, there was a significant difference in the MFIs between PC3-PIP and PC3 cells at the 0.5 h (p<0.01) and 1 h (p<0.01) time points leading to the conclusion that the dendrimer is only taken up and internalized selectively in PSMA (+) cells.

To further study any non-specific uptake of PSMA-2DG-D dendrimer in PSMA (+) cells, a blocking experiment was performed at different time points in the presence a highly PSMA irreversible inhibitor (CTT 1057).²⁸⁻²⁹ In the blocking experiment, a drastic decrease in the uptake of the PSMA-2DG-D dendrimer was observed when PC3-PIP cells were incubated with CTT 1057, a PSMA irreversible inhibitor with nanomolar affinity (FIG. 13B). The MFIs were similar to when the PSMA (−) PC3 cells were treated with the dendrimer. Significant decreases in MFI were observed at all the time points (0.5 h: 8180±494; p<0.001 & 1 h: 7042±1153; p<0.01) when the cells were incubated with CTT 1057 and compared to PC3-PIP cells treated with only the dendrimer. Finally, a dose response study was also performed with different concentrations of PSMA-2DG-D-Cy5 dendrimer to compare the fluorescence of the dendrimer (FIG. 13C). The selective uptake was dose dependent with the highest concentration tested (50 nM) resulting in the highest MFI (29635±2360). A significant increase in MFI was also observed at a lower concentration (10 nM: 5051±393). The lowest 2 concentrations tested, 1 nM (2001±207) and 0.5 nM (2034±152), resulted in no significant difference when compared to the untreated PC3-PIP cells (2287±51).

To further illustrate the trend observed from the flow cytometry experiments, confocal microscopy images were acquired showing the selective uptake in PC3-PIP cells (data not shown). In this experiment, both PC3-PIP (PSMA +) (PSMA −) cells were incubated with 50 μg/mL (2.5 μM) of PSMA-2DG-D-Cy5. To confirm the uptake via PSMA receptors, a blocking experiment was also conducted, where the PC3-PIP cells were incubated with 25 μM of CTT 1057 (10-fold higher concentration compared to the dendrimer) prior to the addition of the dendrimer. As expected, the images showed that the PSMA-2DG-D-Cy5 dendrimer was selectively targeted and taken up by the cancer cells that express high levels of PSMA and internalized rapidly intracellularly following binding to PSMA. A little to no uptake was observed following blocking the PSMA receptor as well as in our negative control PC3 cell line. It is also important to note that minimal uptake was observed in PSMA (−) PC3 cells in these experiments, consistent with previous studies that illustrated the spontaneous uptake and engulfment of non-specific nanoparticles, a common phenomenon observed varying based on the cancer cells phenotype.

We further analyzed the mechanism of internalization of these dendrimers (data not shown). From the above studies, we found that PSMA-2DG-D-Cy5 dendrimers are uptaken by PC3-PIP (PSMA +) cells through ligand binding and in absence of the PSMA there was no uptake, which suggests that these dendrimers are internalizing only by receptor-based endocytosis. Hence, to further decipher the actual mechanism of cell uptake by these PSMA (+) PC3-PIP cells in presence of trafficking inhibitors, confocal laser scanning microscopy was employed. From the results, it was observed that in presence of the CPZ, there was a significant reduction in the fluorescence signal inside the cells as depicted in the micrograph suggesting negligible dendrimer uptake by the cells. In contrast to this, when the cells were treated with MβCD, most of the cells showed presence of red signal (PSMA-2DG-D-Cy5), implying a uptake by the cells. From the overall results, it can be concluded that the PSMA-2DG-D-Cy5 dendrimers are primarily internalized into the PSMA (+) PC3-PIP cells by the clathrin mediated endocytosis pathway, rather than the caveolin mediated one. We further evaluated the internalization mechanism for non-targeted 2DG-D dendrimer as will in PSMA (+) PC3 PIP cells. Recently, we showed that 2DG-D dendrimers are internalized by GLUT receptors present in the neurons. Further, it has been reported in the literature that there is increase ability of prostate cancer cells to acquire glucose and nutrients by facilitative glucose transporters (GLUTs), which are highly expressed in these cancer cells. In PC3 PIP cells, we again found that these dendrimers were positively uptaken by GLUT transporters, as we did not find any red signal (2DG-D-Cy5) when the PC3-PIP (PSMA +) cells were treated with Cytochalasin B, a GLUT transporter inhibitor (data not shown).

### PSMA-2DG-D-Cabo Conjugate Provides Sustained Drug Release at Intracellular Conditions

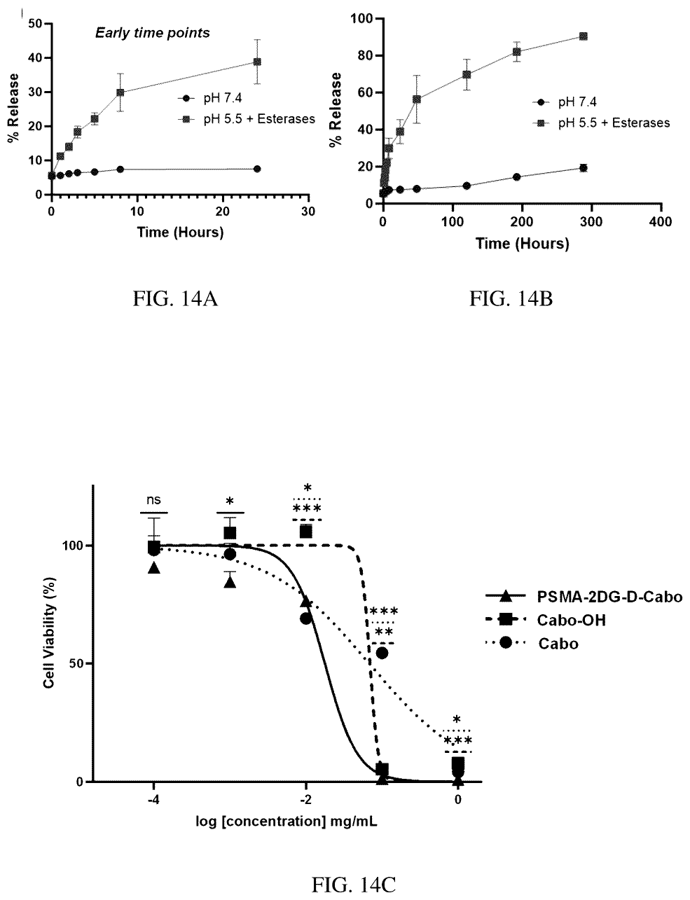

Next, an in vitro investigation was conducted to assess drug release kinetics from the PSMA-2DG-D-Cabo conjugate (FIG. 14A). The experimental conditions simulated both extracellular (physiological pH, phosphate-buffered saline (PBS) at pH 7.4) and intra-tumoral environments (pH 5.5, presence of carboxylesterase). Cabo-OH is conjugated to the dendrimer via an ester bond at its hydroxyl group, positioned at 7, enabling pH and esterase-mediated release. This design enables controlled release at intracellular and intra-tumoral environments, effectively confining drug exposure within the PSMA (+) PC cells. In the PBS buffer at pH 7.4, drug release was limited to less than 20% over a span of approximately two weeks. Notably, only 5% of the drug was released within the initial 8 hours (FIG. 14A). In contrast, under intracellular conditions, the PSMA-2DG-D-Cabo conjugate displayed a gradual and sustained release pattern over the course of two weeks. Approximately 30% of the drug was released within the first 8 hours, gradually escalating to approximately 60% within 48 hours, ultimately achieving an ˜85% drug release over a 12-day period (FIG. 14A-B). This selective and sustained intracellular drug release profile is important to maintain the efficacy of the drug for long time and to avoid off-target effects.

### PSMA-2DG-D-Cabo Conjugate is More Potent than Free Drug in PSMA (+) PC3-PIP Cells

To study the potency, in-vitro efficacy, and targeting of the PSMA-2DG-D-Cabo conjugate, a cell viability experiment was performed in PSMA (+) PC3-PIP cells (FIG. 14C). The dendrimer was investigated and compared with both Cabo and Cabo-OH. PSMA (+) PC3-PIP cells were incubated with different concentrations of the compounds along with the addition of sterile 10% DMSO that served as a control. The cells were incubated for 72 h and their viability was analyzed. As expected, the 10% DMSO led to almost no viable cells present (average (%) of viable cells: 1.04±0.06). PSMA-2DG-D-Cabo was significantly more potent than free Cabo (4.17±0.53; p<0.05) and Cabo-OH (7.81±0.24; p<0.001) at the highest concentration tested, 1000 μg/ml. The average percent of viable cells treated with the highest concentration of the dendrimer conjugate (1.02±0.07) was comparable to the positive (+) control. In fact, dendrimer conjugate was 4 times more potent than free Cabo and more than 7 times more potent than Cabo-OH. A similar trend was observed at 100 μg/ml concentration (Cabo: p<0.001; Cabo-OH: p<0.01). Interestingly, free Cabo was more potent at a lower concentration (69.18±0.94, p<0.05), 10 μg/ml, but the dendrimer conjugate was still more potent (76.57±2.36) when compared to Cabo-OH (105.78±3.09; p<0.001). The dendrimer was also found to be more potent at lower concentrations, specifically at 1 μg/ml (87±4.17) compared to free Cabo (96.34±4.58; p<0.05) and Cabo-OH (105.30±6.38; p<0.05). However, at lowest concentration tested, 0.1 μg/ml, the dendrimer conjugate was comparable to both the free drugs and there was no significant difference observed between the dendrimer and the free drugs, as well as between free Cabo and Cabo-OH. This concentration also had the highest percentage of viable cells present after 72 h comparable to the untreated PC3-PIP cells (average of viable cells normalized to 100%). On a side note, there was no significant differences in potency when free Cabo and Cabo-OH were compared at lower concentrations. However, free Cabo was found to be more potent than Cabo-OH at the higher concentrations tested including 1000 μg/ml (p<0.01) and 10 μg/ml (p<0.01). Overall, the dendrimer conjugate (EC₅₀: 17.70 μg/ml) was more effective at inducing apoptosis in PSMA (+) cells compared to both Cabo (EC₅₀: 68.03 μg/ml) as well as Cabo-OH (EC₅₀: 69.37 μg/ml). This might attribute to the better cellular internalization of PSMA-2DG-D-Cabo than free drugs, higher water solubility, and sustained release profile. Additionally, we also assessed the efficacy of 2DG-D dendrimer platform at varied concentrations against the PSMA (+) PC3-PIP cells. From the results it was clearly observed that there was no significant cytotoxic effect of 2DG-D alone on the PIP cells. As compared to the control non-treated (100%), the cell viability was ˜98, ˜98.5, ˜97.8, ˜97.5, and 96.3% at 0.1, 1, 10, 100 and 1000 μg/ml 2DG-D concentration, respectively (data not shown).

### In Vivo Tumor and Organ Biodistribution of PSMA-2DG-D-Cy5 in Tumor Xenograft Model of Prostate Cancer

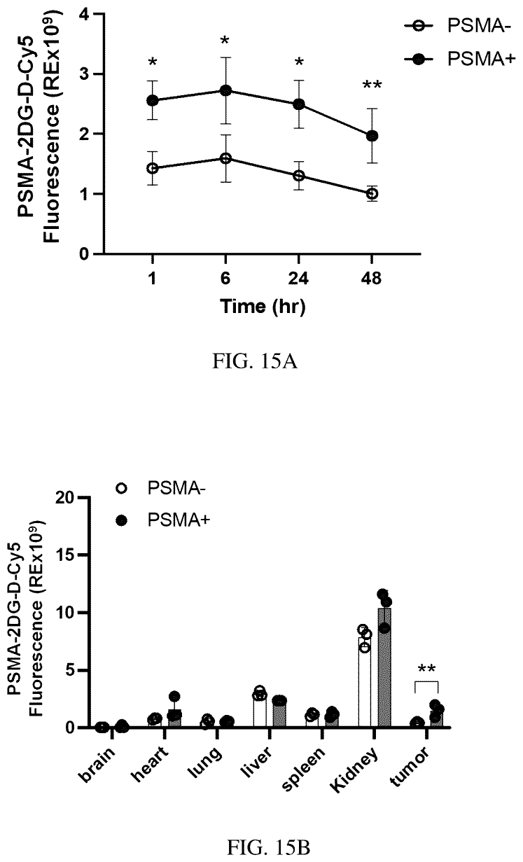

We next assessed if the PSMA-2DG-D-Cy5 retained the PSMA targeting capabilities in vivo (FIG. 15). Before testing the conjugates in vivo, we first evaluated the hemocompatibility of the PSMA-2DG-D-Cy5 dendrimer ex vivo using rat RBCs to ensure their biosafety (data not shown). The hemocompatibility results suggested that the PSMA-2DG-D-Cy5 was biocompatible with the RBCs, showing no signs of haemolysis even at higher concentrations (5 mg/ml) as demonstrated by the UV-Vis spectrum. Further, upon quantitative measurements, the percentage haemolysis was found to be <5% at all tested concentrations [˜4.6% (5 mg/ml), ˜4.3 (2.5 mg/ml), ˜4.5% (1.25 mg/ml), ˜4.4% (0.63 mg/ml), and ˜4.3% (0.31 mg/ml)]. From the above studies, it was observed that at all the concentrations of the PSMA-2DG-D-Cy5; the dendrimers showed excellent compatibility with the rat RBCs. From the above assay results, it can be concluded that, there are no haemolytic effects of PSMA-2DG-D-Cy5 on the RBCs, suggesting them safe for in vivo experiments.

We next evaluated the selective PSMA (+) tumor-targeting capability of PSMA-2DG-D-Cy5 in a human PC3 (PSMA −) and PC3-PIP (PSMA +) tumor xenografts mouse model of PC. PSMA-2DG-D-Cy5 was administered intravenously in PSMA−/+ tumor-bearing mice. Cy5 fluorescence signals were observed and calculated through the in vivo imaging system (IVIS) at 1, 6, 24, and 48 h after injection. PSMA (+) tumors exhibited pronounced fluorescence signals elevation starting as early as 1 h post-injection, persisting significantly above PSMA (−) groups for the duration of the entire 48-h observation period. (FIG. 15A). Moreover, whereas most other nanoparticles, when administered systemically, exhibit unwanted accumulation of up to 80% in the liver, PSMA-2DG-D-Cy5 benefits from its small size (˜4 nm), which falls within the range suitable for renal filtration and clears intact through the kidneys, as previously demonstrated for the 2DG-D nanoplatform. We expect that the dendrimers clear rapidly from kidneys via urine within 24 h after systemic administration, as observed with 2-DG-D-Cy5 dendrimers that showed less than 0.2% 2DG-D-Cy5 in serum after 24 h. The renal clearance has been extensively validated for similarly sized dendrimers of comparable dimensions. The ex vivo fluorescence results at 48 h revealed that the PSMA ligand conjugation on 2DG-D resulted in some non-specific uptake of PSMA-2DG-D-Cy5 primarily in liver, kidneys and spleen (FIG. 15B). A similar non-specific uptake has been shown with small molecule PSMA ligands. Uptake in kidneys is not unusual due to the kidneys being the primary mode for dendrimer clearance. However, significantly higher tumor uptake was observed in PSMA (+) animals compared to PSMA (−) animals at 48 h (FIG. 15B). These results suggest that the PSMA-2DG-D can be a nanoplatform for the delivery of potent chemotherapies, such as Cabo to PSMA (+) prostate tumor, while exhibiting minimum off-target accumulation, and thus less systemic side-effects.

### Conclusions

We designed and developed a PSMA-targeted dendrimer platform (PSMA-2DG-D) for targeted intracellular drug delivery of small molecule therapeutics to PSMA (+) PC cells. The nanoplatform was conveniently synthesized using an efficient chemical strategy via SPAAC chemistry. Our data demonstrates that conjugating an irreversible PSMA inhibitor, CTT-1298, to 2DG-D preserves its PSMA-binding ability at nanomolar concentrations. The PSMA-targeted dendrimer specifically localized in PSMA (+) PC cells via PSMA mediated internalization. We further developed a PSMA-targeted dendrimer cabozantinib conjugate, PSMA-2DG-D-Cabo, which exhibited nanomolar c-Met inhibitory activity and significantly improved anti-proliferative activity in PSMA (+) PC cells compared to the free drug. The PSMA-2DG-D-Cy5 retained its ability to target PSMA (+) PC cells under in vivo conditions in a PC-3 PIP tumor xenograft mouse model. The dendrimer remained in the tumor at least for 48 hours while rapidly clearing from peripheral organs, thereby minimizing systemic side effects. These results suggest that PSMA-targeted dendrimer-based delivery may enhance the efficacy and safety of Cabo and other chemotherapeutic agents for PC treatment, where dose-related systemic toxicities of chemotherapies are a major concern.

### EXAMPLE 3

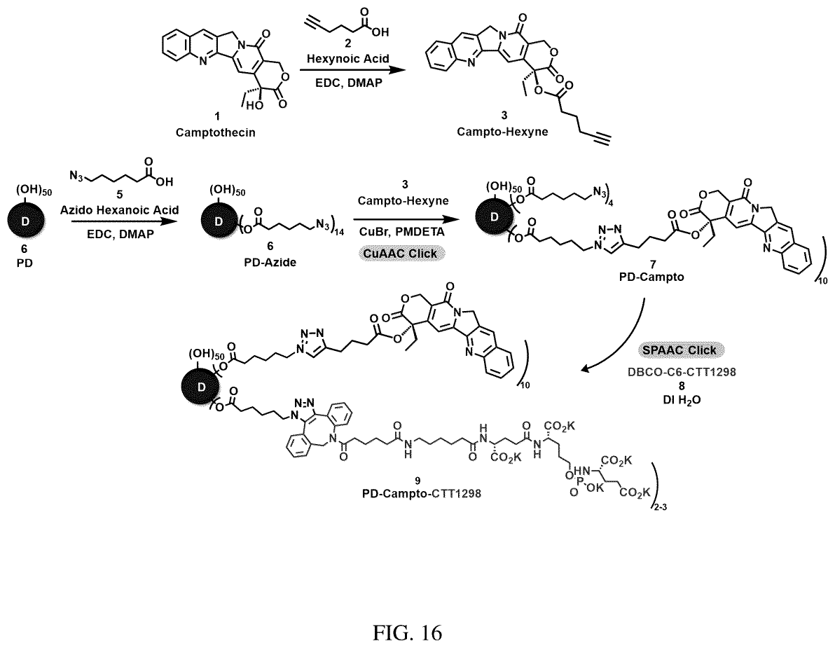

Described below is the synthesis and characterization of a hydroxyl-terminated PAMAM dendrimer conjugated to CTT1298 and camptothecin (FIG. 16).

### Material and Reagents

All starting materials and reagents were obtained from Sigma-Aldrich and Merck. Analytical-grade reagents and solvents used in the synthesis were used as received. Reactions requiring anhydrous conditions were conducted under a positive nitrogen atmosphere, with all glassware dried in an oven beforehand. Analytical thin-layer chromatography (TLC) was performed on silica gel 60 F254 aluminum-backed plates, and spots were visualized using UV light or staining reagents. Compounds were purified by flash column chromatography on silica gel 60 (230-400 mesh). Dialysis membranes (Spectra/Por) were sourced from Repligen.

### Instruments

Nuclear Magnetic Resonance (NMR) spectra were acquired using a Bruker 500 MHZ high-resolution NMR spectrometer, with samples prepared in deuterated solvents such as chloroform (CDCl₃), deuterated DMSO (DMSO-d₆), or deuterated water (D₂O). Chemical shifts for 1H-NMR are given in parts per million (ppm) and are referenced to the solvent peak. Coupling constants (J) are reported in hertz (Hz), with the following abbreviations used to describe signal patterns: s=singlet, d=doublet, t=triplet, q=quartet, and m=multiplet. Microwave reactions were conducted on a Biotage Initiator+instrument using scaled 10 mL or 20 mL process vials, with reaction times referring to the irradiation period at the target temperature, not the total irradiation time; temperature was monitored via an IR sensor.

High-performance liquid chromatography (HPLC) was used to assess the purity of small molecules, as well as dendrimers and dendrimer-drug conjugates, and to evaluate drug release studies. Analyses were performed on a Waters Acquity Arc system (Milford, MA, USA) equipped with binary pumps, a 2998 PDA detector, and a 2475 fluorescence detector, using Waters Empower software. Samples were analyzed on a Waters C18 Symmetry 300 column (5 μm, 4.6×250 mm) with a gradient flow method. The gradient started at 80:20 (Solvent A: 0.1% TFA and 5% ACN in water; Solvent B: 0.1% TFA in ACN), ramped to 60:40 (A:B) over 20 minutes, held at 40:60 (A:B) for 25 minutes, then returned to 80:20 (A:B) at 35 minutes, with a flow rate of 1 mL/min. Dendrimers and drug conjugates were monitored at 210, 254 and 323 nm, while Cy5 conjugates were monitored at 650 nm.

### Synthetic Procedures

Synthesis of Campto-Hexyne (3): To activate, hexynoic acid (2) (96.5 mg, 1.5 eq, 0.86 mmol) was dissolved in anhydrous DMF (5 mL), treated with EDC.HCl (220 mg, 2.0 eq, 1.15 mmol) and stirred for 10-15 minutes. A solution of camptothecin (1) (200 mg, 1.0 eq, 0.57 mmol) in DMF (5 mL) was then added dropwise to the mixture. Following this, DMAP (35 mg, 0.5 eq, 0.29 mmol) was added to the reaction mixture. The reaction was allowed to stir at room temperature for 15 hours, and progress was monitored by thin-layer chromatography (TLC). Upon completion, EtOAc (300 mL) was added, and the organic layer was washed three times with water (3×150 mL) and brine (3×100 mL). The organic phase was dried over anhydrous Na₂SO₄ and concentrated under reduced pressure. The crude was purified by silica gel column chromatography, to get campto-hexyne (3) as a white solid with an 83% yield. HPLC purity: 96.16%, retention time: 20.571 minutes.

¹H NMR (500 MHz, DMSO) δ 8.71 (s, 1H), 8.21-8.10 (m, 2H), 7.88 (ddd, J=8.5, 6.8, 1.5 Hz, 1H), 7.73 (td, J=7.4, 1.2 Hz, 1H), 7.07 (s, 1H), 5.50 (d, J=3.0 Hz, 2H), 5.31 (d, J=3.6 Hz, 2H), 2.86 (t, J=2.7 Hz, 1H), 2.64 (t, J=7.3 Hz, 2H), 2.24 (td, J=7.1, 2.7 Hz, 2H), 2.16 (qd, J=7.1, 2.4 Hz, 2H), 1.74 (pd, J=7.1, 1.9 Hz, 2H), 0.93 (t, J=7.4 Hz, 3H

Synthesis of PD-Azide (6): Azido hexanoic acid (5) (153.9 mg, 14.0 eq, 0.98 mmol) was dissolved in anhydrous DMF (10 mL) and treated with EDC.HCl (376.54 mg, 28.0 eq, 1.96 mmol), then stirred for 10-15 minutes. A solution of PAMAM G4 OH dendrimer (PD) (4) (1 g, 1.0 eq, 0.07 mmol) in DMF (5 mL) was then added dropwise to the activated hexynoic acid solution. DMAP (59.8 mg, 7.0 eq, 0.49 mmol) was subsequently introduced to the mixture, which was allowed to stir at room temperature for 12 hours. Completion was confirmed by HPLC analysis, indicated by a shift in the retention time of the starting material. Dialysis was carried out using a 1 kDa membrane in DMF for 12 hours, followed by a final dialysis in DI water. The product was lyophilized, to afford PD-Azide (6) with an 86% yield. HPLC purity: 99.66%, retention time: 14.410 minutes.

¹H NMR (500 MHz, DMSO) δ 8.13-7.66 (m, 124H), 4.89-4.57 (m, 49H), 3.99 (p, J=5.7 Hz, 23H), 3.49-3.23 (m, 187H), 3.10 (tt, J=11.2, 5.5 Hz, 205H), 2.89 (s, 18H), 2.73 (dd, J=10.9, 5.0 Hz, 55H), 2.52-2.36 (m, 136H), 2.32-2.11 (m, 253H), 1.53 (dp, J=14.6, 7.1 Hz, 49H), 1.38-1.24 (m, 24H).

Synthesis of PD-Campto (7): To a stirred solution of compound 6 (150 mg, 1.0 eq, 0.009 mmol) in 1 mL DI water was added solution of compound 3 (42 mg, 10.0 eq, 0.09 mmol) dissolved in 1 mL DMF followed by the addition of CuSO₄.5H₂O (10 mol % per acetylene) dissolved in 0.1 mL DI water. After 2 minutes of stirring, sodium ascorbate (15 mol % per acetylene) was added, and the reaction mixture was stirred at 40° C. for 16 h. Upon completion, the compound was purified on TFF using a 3 kDa dialysis membrane. The aqueous solution was lyophilized to afford compound 7 in 90% yield. HPLC purity: 99.47%, retention time: 16.095 minutes.

¹H NMR (500 MHz, DMSO) δ 8.60 (s, 7H), 8.11-7.61 (m, 194H), 6.97 (d, J=28.0 Hz, 12H), 5.32 (d, J=106.1 Hz, 20H), 4.66 (s, 68H), 4.17 (s, 20H), 3.91 (s, 26H), 3.32 (s, 121H), 3.04 (s, 251H), 2.58 (s, 311H), 2.36 (s, 111H), 2.13 (s, 362H), 1.74 (d, J=68.8 Hz, 64H), 1.44 (s, 49H), 1.30-1.05 (m, 49H), 0.97-0.79 (m, 35H).

Synthesis of PD-Campto-CTT1298 (8): A stirred solution of compound 7 (150 mg, 1.0 eq, 0.0075 mmol) in 1 mL DI water was treated with a solution of DBCO-C6-CTT1298 (19.34 mg, 2.5 eq, 0.018 mmol) in 1 mL DI water and the reaction mixture stirred at room temperature for 8 hours. Upon completion, the mixture was dialyzed with a 1 kDa membrane in DI water for 12 hours. The resulting solution was lyophilized, yielding compound 8 with an 87% yield. HPLC purity: 99.30%, retention time: 16.074 minutes.

¹H NMR (500 MHz, DMSO) δ 8.73 (s, 11H), 8.25-7.69 (m, 320H), 7.32 (s, 28H), 7.16-6.96 (m, 31H), 5.55 (s, 27H), 5.34 (s, 24H), 4.80 (s, 80H), 4.30 (s, 62H), 4.09-3.98 (m, 61H), 3.65-3.30 (m, 712H), 3.26-3.02 (m, 468H), 2.90-2.62 (m, 582H), 2.49 (s, 178H), 2.26 (s, 500H), 2.02-1.73 (m, 76H), 1.55 (d, J=17.8 Hz, 48H), 1.27 (d, J=22.1 Hz, 66H), 1.07-0.89 (m, 36H). ³¹P NMR (202 MHz, DMSO) δ 6.86.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. A dendrimer complex comprising
a dendrimer; and
a prostate specific membrane antigen (PSMA) ligand conjugated to an outer surface of the dendrimer, wherein the PSMA ligand is CTT1298 or ACUPA.

2. The dendrimer complex of claim 1, wherein the dendrimer is a generation 0-10 dendrimer.

3. The dendrimer complex of claim 1, wherein the dendrimer is a polyamidoamine (PAMAM) dendrimer.

4. The dendrimer complex of claim 1, wherein the dendrimer is a 2-deoxyglucose (2DG) dendrimer.

5. The dendrimer complex of claim 1, wherein the complex further comprises one or more drugs, imaging agents, and/or radioligands conjugated to an outer surface of the dendrimer.

6. The dendrimer complex of claim 5, wherein the one or more drugs comprises an anti-cancer small molecule drug.

7. The dendrimer complex of claim 6, wherein the anti-cancer small molecule drug is cabozantinib or hydroxyl-cabozanitib.

8. The dendrimer complex of claim 6, wherein the anti-cancer small molecule drug is camptothecin.

9. A pharmaceutical composition comprising the dendrimer complex of claim 1 and a pharmaceutically acceptable carrier.

10. A method of inhibiting proliferation of PMSA-expressing cells, comprising contacting the PMSA-expressing cells with the dendrimer complex of claim 6.

11. A method of treating cancer characterized by expression of PMSA in a subject in need thereof, comprising administering to the subject a therapeutically effective amount of the dendrimer complex of claim 6.

12. The method of claim 11, wherein the cancer is prostate cancer.

13. A method of detecting PMSA-expressing cells, comprising
contacting the PMSA-expressing cells with the dendrimer complex of claim 1, wherein the dendrimer complex further comprises an imaging agent conjugated to an outer surface of the dendrimer; and
detecting the imaging agent.

14. A dendrimer complex comprising
a hydroxyl-terminating polyamidoamine (PAMAM) dendrimer having a neutral surface charge; and
a prostate specific membrane antigen (PSMA) ligand conjugated to an outer surface of the dendrimer, wherein the PSMA ligand is not 2-PMPA.

15. The dendrimer complex of claim 14, wherein the dendrimer is a generation 0-10 dendrimer.

16. The dendrimer complex of claim 14, wherein the complex further comprises one or more drugs, imaging agents, and/or radioligands conjugated to an outer surface of the dendrimer.

17. The dendrimer complex of claim 16, wherein the one or more drugs comprises an anti-cancer small molecule drug.

18. A dendrimer complex comprising
a 2-deoxyglucose (2DG) dendrimer; and
a prostate specific membrane antigen (PSMA) ligand conjugated to an outer surface of the dendrimer.

19. The dendrimer complex of claim 18, wherein the complex further comprises one or more drugs, imaging agents, and/or radioligands conjugated to an outer surface of the dendrimer.

20. The dendrimer complex of claim 19, wherein the one or more drugs comprises an anti-cancer small molecule drug.
